(19) United States
(12) Patent Application Publication
(10) Pub. No.: US 2025/0215469 A1
(43) Pub. Date: Jul. 3, 2025

(54) METHOD FOR PRODUCING SESAMINOL OR SESAMINOL GLUCOSIDE

(71) Applicant: FLAVOR FULL FOODS INC., Taipei City (TW)

(72) Inventors: NAN-WEI SU, Taipei City (TW); YI-CHEN LO, Taipei City (TW); CHANG-HUNG CHEN, Taipei City (TW); CHAO-YANG HSU, Taipei City (TW)

(21) Appl. No.: 19/003,571

(22) Filed: Dec. 27, 2024

(30) Foreign Application Priority Data
Dec. 27, 2023 (TW) 112151080

(51) Int. Cl. C12P 17/18; C12N 9/24; C12N 15/62; C12P 19/60

(52) U.S. Cl. CPC C12P 17/181; C12N 9/2405; C12N 15/62; C12P 19/60

(57) ABSTRACT

The present invention provides a method for producing sesaminol or sesaminol glucosides comprising: reacting a protein with a substrate sesaminol glycoside having at least one glycosidic bond, and catalyzing the hydrolysis of the glycosidic bond; wherein, the protein is selected from the group consisting of the following (1) to (3): (1) a protein composed of the amino acid sequence SEQ ID NO: 1; (2) a protein composed of the amino acid sequence formed by deletion, substitution, insertion and/or addition of one or more amino acids in the amino acid sequence SEQ ID NO:1, wherein the protein has the activity of catalyzing the hydrolysis of the glycosidic bond; (3) a protein composed of an amino acid sequence having an sequence identity of more than 60% compared with the amino acid sequence of SEQ ID NO: 1, wherein the protein has the activity of catalyzing the hydrolysis of the glycosidic bond. Said method of the present invention can not only shorten the process time, but also increase the yield of sesaminol and reduce the production cost, thereby meeting the needs of industrial applications.

### REFERENCE TO AN ELECTRONIC SEQUENCE LISTING

The content of the electronic sequence listing (2024-03-24-Sequence-Listing.xml; Size: 15 KB; and Date of Creation: Mar. 24, 2025) is herein incorporated by reference in its entirety.

### BACKGROUND OF THE INVENTION

### 1. Technical Field

The present invention relates to a method for generating a compound, and more particularly to a method for generating sesaminol or sesaminol glucosides. However, the present invention is not limited thereto.

### 2. Description of Related Art

Sesame (Sesamum indicum L), a sesamum plant of the family Pedaliaceae, has a long history of cultivation. In early days, it was mainly used as food or for medical treatment. Studies have shown that lignans are the main physiologically active ingredients in sesame, and are also found in the processing by-products of sesame.

Lignans are secondary metabolites of plants, which are formed by the oxidative coupling of p-hydroxyphenyl propane of two molecules. They can be divided by water solubility into two categories, among which oil-soluble lignans include sesamin, sesamolin, sesamol, sesaminol, sesamolinol, and pinoresinol, etc., which are mainly found in sesamen oil. In addition, the lignans exist in the form of glucosides, and lignan glucosides include sesaminol glucosides (SGs) and pinoresinol glucosides, etc.

In recent years, sesaminol has been found to have stronger antioxidant activity than that of other sesame lignans, and it can reduce the occurrence of Alzheimer's disease or prevent Parkinson's disease. However, sesaminol has a low content in sesame pressed meal and exists mainly in the form of glucosides. Therefore, it is an important topic in this field to efficiently hydrolyze sesaminol glucosides to obtain sesaminol.

Common methods for producing sesaminol involve hydrolysis of degreased sesame pressed meal with commercial enzymes. However, this production process is time-consuming and requires the addition of a large amount of enzymes, which is not cost-effective from a commercial point of view.

### BRIEF SUMMARY OF THE INVENTION

In the acquired technologies, common methods for producing sesaminol involve hydrolysis of degreased sesame pressed meal with commercial enzymes. However, this production process is time-consuming and requires a large amount of enzymes, which is not cost-effective from a commercial point of view. In view of this, the present invention aims to provide a method for effectively generating a large amount of sesaminol, whereby the processing time can be reduced to decrease the cost and then improve commercial benefits. In the present application, the inventor of the present invention found that an enzyme Kmbgl1 can be used for hydrolysis of a glucosidic bond in sesaminol glucosides to generate sesaminol. Based on this, the inventor established a method for effectively producing sesaminol by taking a crude sesame extract as a starting material.

Specifically, in one aspect, the present invention provides a method for generating sesaminol or sesaminol glucosides, including the steps of: reacting a protein with a substrate sesaminol glycoside having at least one glucosidic bond, and catalyzing hydrolysis of at least one glucosidic bond. Wherein, the protein is selected from the group consisting of (1) to (3): (1) a protein that comprises an amino acid sequence of SEQ ID NO: 1; (2) a protein that comprises an amino acid sequence derived by deletion, substitution, insertion and/or addition of one or more amino acids in the amino acid sequence of SEQ ID NO: 1 and has an activity of catalyzing the hydrolysis of at least one glucosidic bond; and (3) a protein that comprises an amino acid sequence having more than 60% sequence identity to the amino acid sequence of SEQ ID NO: 1 and has an activity of catalyzing the hydrolysis of at least one glucosidic bond.

According to an embodiment of the present invention, the substrate sesaminol glycoside is selected from the group consisting of: sesaminol 2′-O-β-D-glucopyranoside (sesaminol monoglucoside, SMG), sesaminol 2′-O-β-D-glucopyranosyl(1-2)-O-β-D-glucopyranoside (sesaminol (1-2)diglucoside, SDG (1,2)), sesaminol 2′-O-β-D-glucopyranosyl(1-6)-O-β-D-glucopyranoside (sesaminol (1-6)diglucoside, SDG (1,6)) and sesaminol 2′-O-β-D-glucopyranosyl(1-2)-O-(-β-D-glucopyranosyl(1-2))β-D-glucopyranoside (sesaminol triglucoside, STG).

According to an embodiment of the present invention, the substrate sesaminol glycoside is a 60% (v/v) methanol crude extract.

According to an embodiment of the present invention, the sesaminol or sesaminol glucosides is selected from the group consisting of: sesaminol (1-6)diglucoside (SDG (1,6)), sesaminol (1-2)diglucoside (SDG (1,2)) and sesaminol.

According to an embodiment of the present invention, the temperature for reacting the protein with the substrate sesaminol glycoside is 37° C. to 45° C.

According to an embodiment of the present invention, the reaction of the protein with the substrate sesaminol glycoside occurs at pH 5.5 to pH 6.5.

According to an embodiment of the present invention, the reaction time for the protein with the substrate sesaminol glycoside is 16 hours.

According to an embodiment of the present invention, at least one of glucosidic bond is selected from the group consisting of: a glycosidic bond bonding between glucose bonded to position 2′ of sesaminol and an aglycone, a β-1,6-glucosidic bond bonding to gentiobiose at position 2′ of sesaminol, a β-1,2 bond bonding to sophoroze at position 2′ of sesaminol, and a β-1,6-glucosidic bond and a β-1,2 bond bonding of branched triglucoside at position 2′ of sesaminol.

In another aspect, the present invention provides a method for generating sesaminol or sesaminol glucosides, comprising the steps of: in a host cell, an enzyme from a non-human transformed cell is reacted with substrate sesaminol glycoside having at least one glucosidic bond, and catalyzing hydrolysis of at least one glucosidic bond. Wherein, a polynucleotide is introduced into the non-human transformed cell, and the polynucleotide is selected from the group consisting of (1) to (5): (1) a polynucleotide encoding a protein that comprises an amino acid sequence of SEQ ID NO: 1; (2) a polynucleotide encoding a protein that comprises an amino acid sequence derived by deletion, substitution, insertion and/or addition of one or more amino acids in the amino acid sequence of SEQ ID NO: 1 and has an activity of catalyzing the hydrolysis of the at least one glucosidic bond; (3) a polynucleotide encoding a protein that comprises an amino acid sequence having more than 60% sequence identity to the amino acid sequence of SEQ ID NO: 1 and has an activity of catalyzing the hydrolysis of at least one glucosidic bond; (4) a polynucleotide encoding a protein that has an activity of catalyzing the hydrolysis of at least one glucosidic bond, which hybridizes with a polynucleotide comprising a complementary base sequence under a highly demanding condition, wherein the complementary base sequence is complementary to the polynucleotide encoding the protein comprising the amino acid sequence of SEQ ID NO: 1; and (5) a polynucleotide comprising the base sequence of SEQ ID NO:2.

According to an embodiment of the present invention, the polynucleotide is inserted into an expression vector.

According to an embodiment of the present invention, the non-human transformed cell is selected from the group consisting of: a transformed plant cell, a transformed animal cell, a transformed insect cell, transformed Escherichia coli, transformed Bacillus subtilis, transformed Actinomycetes, transformed bacteria, transformed Saccharomyces, and transformed Filamentous bacteria.

According to an embodiment of the present invention, the substrate sesaminol glycoside is selected from the group consisting of: sesaminol 2′-O-β-D-glucopyranoside (sesaminol monoglucoside, SMG), sesaminol 2′-O-β-D-glucopyranosyl(1-2)-O-β-D-glucopyranoside (sesaminol (1-2)diglucoside, SDG (1,2)), sesaminol 2′-O-β-D-glucopyranosyl(1-6)-O-β-D-glucopyranoside (sesaminol (1-6)diglucoside, SDG (1,6)), and sesaminol 2′-O-β-D-glucopyranosyl(1-2)-O-(-β-D-glucopyranosyl(1-2))β-D-glucopyranoside (sesaminol triglucoside, STG).

According to an embodiment of the present invention, the sesaminol or sesaminol glucosides is selected from the group consisting of: sesaminol (1-6)diglucoside (SDG (1,6)), sesaminol (1-2)diglucoside (SDG (1,2)) and sesaminol.

According to an embodiment of the present invention, at least one of glucosidic bond is selected from the group consisting of: a glycosidic bond bonding between glucose bonded to position 2′ of sesaminol and an aglycone, a β-1,6-glucosidic bond bonding to gentiobiose at position 2′ of sesaminol, a β-1,2 bond bonding to sophoroze at position 2′ of sesaminol, and a β-1,6-glucosidic bond and a β-1,2 bond bonding to branched triglucoside at position 2′ of sesaminol.

The method provided by the present invention has the following advantages: shorted processing time, improved yield of sesaminol and reduced manufacture cost, which meets the application requirements in industry.

### BRIEF DESCRIPTION OF THE DRAWINGS

The implementations of the technology of the present invention are described now with reference to the accompanying drawings by way of example. In the accompanying drawings:

FIG. 1 shows the analysis results of a Kmbgl1 protein purified by Histrap™HP columns according to an embodiment of the present invention, with M in the figure representing a molecular weight standard, CE representing a crude cell extract, E1 to E8 representing proteins eluted with 200 mM imidazole by means of different columns;

FIGS. 2A-2D show the HPLC profile results of a sesame pressed meal extract hydrolyzed with an enzyme Kmbgl1 for (A) 0, (B) 1, (C) 8, and (D) 24 hours according to an embodiment of the present invention, where as indicated, 1a, 1b, 1c, and 1d correspond to peaks of STG (Rt 10.5-11.5 min, [M⁺H⁺] m/z 857.27), 2a, 2b, 2c, and 2d correspond to peaks of SDG (Rt 12.3-13.8 min, [M′H⁺] m/z 695.27), and 3a, 3b, 3c, 3d correspond to peaks of sesaminol (Rt 20.0-20.6, 21.5-22.3 min, [M⁺H⁺] m/z 371.11);

FIG. 3 shows the effects of enzymes added in different amounts on the generation of sesaminol glucosides according to an embodiment of the present invention;

FIG. 4 shows the effects of different reaction time on the generation of sesaminol glucosides according to an embodiment of the present invention;

FIG. 5 shows the effects of different pH values on the generation of sesaminol glucosides according to an embodiment of the present invention;

FIG. 6 shows the effects of different reaction temperatures on the generation of sesaminol glucosides according to an embodiment of the present invention, with (A) showing the results under condition of 37° C., (B) showing the results under condition of 40° C., and (C) showing the results under condition of 45° C.;

FIG. 7 shows the measurements of the content of sesaminol glucosides obtained by reacting the hot-water extract of sesame pressed meal with the crude extract of enzyme liquids of different Saccharomyces according to an embodiment of the present invention;

FIG. 8 shows a flow chart of preparing sesaminol with a 60% methanol extract according to an embodiment of the present invention; and

FIG. 9 shows a flow chart of preparing sesaminol with a water extract according to an embodiment of the present invention.

It should be understood that the present invention is not limited to the configurations, means, and characteristics shown in the accompanying drawings in all aspects.

### DETAILED DESCRIPTION OF THE INVENTION

The implementations below should not be considered as overly limiting the present invention. Without departing from the spirit or scope of the present invention, a person of ordinary skill in the art in the technical field to which the present invention belongs can make modifications and variations to the embodiments discussed herein, and these modifications and variations shall still fall within the scope of the present invention.

Unless otherwise specified in the context, the terms “comprise,” “include,” “have”, or “contain” are inclusive or open-ended, and do not exclude other unstated elements or method steps; the terms “a/an” and “the” can be interpreted as either singular or plural; and the term “one or more” means “at least one”, and thus can include a single feature or a mixture/combination.

The numerical values or experimental data provided herein are substantially approximate numerical values, and relevant numerical values are presented here in the specific embodiments as accurate as possible. However, any numerical value in essence inevitably contains a standard deviation due to individual test methods. Here, the word “approximate” represents the actual value falling within the acceptable standard error of the mean, and this is determined by consideration of a person of ordinary skill in the art to which the present invention belongs.

The present invention provides a method for generating sesaminol or sesaminol glucosides, including the steps of: reacting a protein with a substrate sesaminol glucosides having at least one glucosidic bond, and catalyzing hydrolysis of at least one glucosidic bond. The “sesaminol glucosides (SGs)” described herein refer to glucosides derived by bonding sesaminol to glucose, and they are also referred to as sesaminol glucosides.

According to an embodiment of the present invention, the protein is selected from the group consisting of (1) to (3): (1) a protein that comprises an amino acid sequence of SEQ ID NO: 1 (MSKFDVEQLLSELNQDEKISLLSAVDFWHTKKIERLGIPAVRVSDGPNGIRGTKFF DGVPSGCFPNGTGLASTFDRDLLETAGKLMAKESIAKNAAVILGPTTNMQRGPLG GRGFESFSEDPYLAGMATSSVVKGMQGEGIAATVKHFVCNDLEDQRFSSNSIVSE RALREIYLEPFRLAVKHANPVCIMTAYNKVNGEHCSQSKKLLIDILRDEWKWDG MLMSDWFGTYTTAAAIKNGLDIEFPGPTRWRTRALVSHSLNSREQITTEDVDDRV RQVLKMIKFVVDNLEKTGIVENGPESTSNNTKETSDLLRKIAADSIVLLKNKNNIL PLKKEDNIIVIGPNAKAKTSSGGGSASMNSYYVVSPYEGIVNKLGKEVDYTVGAY SHKSIGGLAESSLIDAAKPADAENSGLIAKFYSNPVEERSDDEEPFHVTKVNRSNV HLFDFKHEKVDPKNPYFFVTLTGQYVPQEDGDYIFSLQVYGSGLFYLNDELIIDQK HNQERGSFCFGAGTKERTKKLTLKKGQVYNVRVEYGSGPTSGLVGEFGAGGFQA GVIKAIDDDEEIRNAAELAAKHDKAVLIIGLNGEWETEGYDRENMDLPKRTNELV RAVLKANPNTVIVNQSGTPVEFPWLEDANALVQAWYGGNELGNAIADVLYGDV VPNGKLSLSWPFKLQDNPAFLNFKTEFGRVIYGEDIFVGYRYYEKLQRKVAFPFG YGLSYTTFELDISDFKVTDDKIAISVDVKNTGDKFAGSEVVQVYFSALNSKVSRPV KELKGFEKVHLEPGEKKTVNIDLELKDAISYFNEELGKWHVEAGEYLVSVGTSSD DILSVKEFKVEKELYWKGL); (2) a protein that comprises an amino acid sequence derived by deletion, substitution, insertion and/or addition of one or more amino acids in the amino acid sequence of SEQ ID NO: 1 and has an activity of catalyzing the hydrolysis of the at least one glucosidic bond; and (3) a protein that comprises an amino acid sequence having more than 60% sequence identity to the amino acid sequence of SEQ ID NO: 1 and has an activity of catalyzing the hydrolysis of the at least one glucosidic bond.

The sequence of SEQ ID NO: 1 is derived from Kluyveromyces marxianus. Kluyveromyces marxianus belongs to fungi and is Saccharomyces. According to at least one embodiment of the present invention, the protein comprising the amino acid sequence of SEQ ID NO: 1 is an enzyme Kmbgl1 derived from the Saccharomyces described above. Specifically, the protein structurally comprises four parts, namely, (α/β) 8 barrel-like (corresponding positions 1 to 295 of SEQ ID NO: 1), a PA14 domain (corresponding to positions 392-559), (a/B) 6 sandwich (corresponding to positions 307 to 381 and 560 to 658) and a C-terminal domain (corresponding to positions 700-845), belongs to the glycosidase hydrolase family 3 (GH3), which is capable of hydrolyzing sesaminol triglucoside (STG) or sesaminol diglucoside (SDG) to generate the sesaminol and sesaminol glucosides. The active sites of Kmbgl1 include D225 and E590, as well as several glucose binding sites, for example, D45, L99, R113, K146, H147, R157, M190, Y193, D225, W266, S356, F445, F508, E590, etc.

According to at least one embodiment of the present invention, the enzyme of the present invention employs a protein comprising the amino acid sequence of SEQ ID NO: 1 or a variant thereof; and the variant comprises those derived by artificial means. Furthermore, the “amino acid sequence derived by deletion, substitution, insertion and/or addition of one or more amino acids” may be derived, for example, by deletion, substitution, insertion, and/or addition of 1 to 3, 1 to 5, 1 to 10, 1 to 20, 1 to 50, and 1 to 80 amino acids; and in general, the number of the amino acid deleted, substituted, inserted and/or added is preferably small. In addition, the amino acid sequence having more than 60% sequence identity is, but not limited to, an amino acid sequence having more than 60%, more than 65%, more than 70%, more than 75%, more than 80%, more than 85%, more than 90%, more than 97%, more than 98%, or more than 99% sequence identity; and preferably, the amino acid sequence has more than 75% sequence identity.

The “substrate sesaminol glucosides” of the present invention refers to sesaminol glucosides as a reaction substrate. According to an embodiment of the present invention, the substrate sesaminol glucosides is selected from the group consisting of: sesaminol 2′-O-β-D-glucopyranoside (sesaminol monoglucoside, SMG), sesaminol 2′-O-β-D-glucopyranosyl(1-2)-O-β-D-glucopyranoside (sesaminol (1-2)diglucoside,SDG (1,2)); sesaminol 2′-O-β-D-glucopyranosyl(1-6)-O-β-D-glucopyranoside (sesaminol (1-6)diglucoside,SDG (1,6)), and sesaminol 2′-O-β-D-glucopyranosyl(1-2)-O-(-β-D-glucopyranosyl(1-2))β-D-glucopyranoside (sesam inol triglucoside,STG). Preferably, the sesaminol glucosides substrate is sesaminol triglucoside (STG). According to an implementation of the present invention, rarer sesaminol glucosides such as SDG (1,2) or SDG (1,6) can be generated by reacting with the enzyme provided by the present invention with most content abundant of the sesaminol glucosides in sesame pressed meal as a substrate.

The at least one glucosidic bond of the sesaminol glucosides refers to the glucosidic bond (also referred to a glucosidic bond between sesaminol and a side chain in the sesaminol glucosides, as well as a glucosidic bond within the side chain. Furthermore, the “activity of hydrolyzing the glucosidic bond of the substrate sesaminol glucosides having at least one glucosidic bond” refers to an activity of hydrolyzing (cleavage) at least one glucosidic bond in the sesaminol glucosides. According to an embodiment of the present invention, at least one glucosidic bond is selected from the group consisting of: a glucosidic bond bonding between glucose bonded to position 2′ of sesaminol and an aglycone, a β-1,6-glucosidic bond bonding to gentiobiose at position 2′ of sesaminol, a β-1,2 bond bonding to sophoroze at position 2′ of sesaminol, and a β-1,6-glucosidic bond and a β-1,2 bond bonding to branched triglucoside at position 2′ of sesaminol. According to an embodiment of the present invention, hydrolyzing the glucosidic bond refers to complete hydrolysis of the glucosidic bond to produce sesaminol from the sesaminol glucosides. In another implementation, preferably, a β-1,6-glucosidic bond that bonds to a branched triglucoside at position 2′ of sesaminol is hydrolyzed. Accordingly, the method for generating the sesaminol or sesaminol glucosides of the present invention can be used to generate the sesaminol glucosides by cutting off only part of the glucosidic bond, or to generate the sesaminol by cutting off the glucosidic bond completely.

In addition, the inventor of the present application has found that at least one of the amount of the protein added, the method for extracting the substrate, the reaction temperature, the reaction pH value and the reaction time has further affected on the generation of the sesaminol or sesaminol glucosides.

According to a preferred embodiment of the present invention, the substrate sesaminol glucosidesis 60% (v/v) methanol crude extract. Specifically, the 60% (v/v) methanol crude extract is obtained by extracting sesame pressed meal with 60% (v/v) methanol at room temperature.

According to a preferred embodiment of the present invention, the temperature for reacting the protein with the substrate sesaminol glucosides is 37° C. to 45° C., for example, but not limited to, any one of or the one between any two of: 37° C., 38° C., 39° C., 40° C., 41° C., 42° C., 43° C., 44° C., and 45° C.; and preferably, the temperature for reacting the protein with the substrate sesaminol glucosides is 40° C.

According to a preferred embodiment of the present invention, the reaction of the protein with the substrate sesaminol glucosides occurs at pH 5.5 to pH 6.5, for example, but not limited to, any one of or the one between any two of: pH 5.5, pH 5.6, pH 5.7, pH 5.8, pH 5.9, pH 6.0, pH 6.1, pH 6.2, pH 6.3, pH 6.4, and pH 6.5; and preferably, reaction of the protein with the substrate sesaminol glucosides occurs at pH 6.5.

According to a preferred embodiment of the present invention, the reaction time for the protein with the substrate sesaminol glucosides is 12 hours to 16 hours, for example, but not limited to, any one of or the one between any two of: 12 hours, 13 hours, 14 hours, 15 hours, and 16 hours; and preferably, the reaction time for the protein with the substrate sesaminol glucosides is 16 hours.

In another aspect, the present invention provides a method for generating sesaminol or sesaminol glucosides, comprising the steps of: in a host cell, an enzyme from a non-human transformed cell is reacted with substrate sesaminol glycoside having at least one glucosidic bond, and catalyzing hydrolysis of at least one glucosidic bond.

According to an embodiment of the present invention, a polynucleotide is introduced into the non-human transformed cell, and the polynucleotide is selected from the group consisting of (1) to (5): (1) a polynucleotide encoding a protein that comprises an amino acid sequence of SEQ ID NO: 1; (2) a polynucleotide encoding a protein that comprises an amino acid sequence derived by deletion, substitution, insertion and/or addition of one or more amino acids in the amino acid sequence of SEQ ID NO: 1 and has an activity of catalyzing the hydrolysis of the at least one glucosidic bond; (3) a polynucleotide encoding a protein that comprises an amino acid sequence having more than 60% sequence identity to the amino acid sequence of SEQ ID NO: 1 and has an activity of catalyzing the hydrolysis of the at least one glucosidic bond; (4) a polynucleotide encoding a protein that has an activity of catalyzing the hydrolysis of the at least one glucosidic bond, which hybridizes with a polynucleotide comprising a complementary base sequence at a highly demanding condition, wherein the complementary base sequence is complementary to the polynucleotide encoding the protein comprising the amino acid sequence of SEQ ID NO: 1; and (5) a polynucleotide comprising a base sequence of SEQ ID NO: 2 (ATGTCTAAATTTGATGTTGAACAGTTATTGAGTGAATTGAACCAGGATG AAAAGATTTCCTTACTCTCTGCAGTTGATTTCTGGCATACTAAGAAGATTGAA CGGTTGGGAATTCCAGCGGTGAGGGTTTCTGATGGTCCAAATGGTATTAGAGG GACAAAGTTCTTTGATGGGGTTCCTTCAGGATGTTTCCCTAATGGTACCGGGTT GGCATCTACTTTTGATCGCGACCTGCTTGAGACAGCAGGTAAGTTGATGGCCA AGGAATCGATTGCGAAGAATGCTGCTGTGATTTTGGGTCCAACCACAAACATG CAACGTGGTCCTTTGGGTGGTCGTGGTTTTGAATCATTTTCTGAAGATCCATAC CTTGCTGGTATGGCAACTTCTTCTGTTGTTAAAGGTATGCAGGGCGAAGGTAT TGCTGCTACCGTTAAGCATTTTGTTTGTAACGATTTGGAAGACCAACGTTTCTC TTCAAACTCAATTGTTTCTGAAAGGGCTCTTAGAGAAATTTACTTGGAGCCCTT CAGATTGGCAGTTAAACATGCCAATCCTGTTTGTATAATGACTGCCTATAACA AGGTCAATGGCGAACATTGCTCCCAATCCAAGAAGCTATTGATCGACATTTTG AGAGACGAGTGGAAATGGGACGGTATGTTAATGTCCGACTGGTTCGGTACAT ATACGACTGCCGCAGCTATCAAGAATGGGTTGGATATCGAGTTTCCTGGACCA ACAAGATGGAGAACACGTGCTTTAGTGTCTCACTCACTCAACTCCAGAGAACA AATCACTACTGAAGATGTTGATGATCGTGTTAGACAAGTGCTAAAAATGATTA AGTTCGTTGTTGACAATTTAGAGAAAACAGGTATTGTGGAGAATGGCCCAGA ATCTACTTCAAACAACACCAAGGAAACCTCGGACCTGTTGAGAAAGATTGCTG CTGACTCTATTGTTTTATTGAAGAACAAAAACAATATCTTACCTCTAAAGAAA GAAGACAATATCATTGTCATTGGCCCAAATGCTAAAGCAAAGACTAGTTCTGG TGGTGGTTCAGCATCCATGAACTCCTACTATGTCGTTTCTCCGTATGAAGGTAT CGTCAATAAGCTGGGCAAAGAGGTCGACTACACCGTAGGCGCCTATTCACAC AAATCTATTGGAGGTTTGGCCGAGAGTAGTTTGATCGATGCTGCAAAACCAGC AGATGCTGAAAATTCTGGATTAATTGCCAAGTTTTACTCCAATCCAGTAGAAG AGAGATCTGACGATGAAGAACCATTCCACGTTACCAAAGTCAATAGATCCAA TGTTCACTTATTTGATTTCAAACATGAGAAAGTGGATCCAAAGAACCCTTACT TTTTTGTAACCTTAACCGGACAGTACGTGCCCCAAGAAGATGGTGATTATATC TTCAGTCTTCAAGTTTATGGTTCTGGTTTGTTCTACTTAAACGATGAGTTGATT ATTGACCAAAAGCACAACCAAGAAAGGGGTAGTTTCTGCTTTGGAGCTGGTA CCAAAGAAAGAACCAAAAAGTTGACTTTGAAGAAGGGCCAAGTTTATAATGT AAGAGTTGAGTACGGTTCTGGCCCAACTTCAGGTTTGGTTGGGGAATTCGGTG CAGGTGGATTCCAAGCTGGTGTCATCAAGGCCATCGACGATGACGAGGAGAT TAGAAACGCAGCGGAATTAGCAGCTAAGCATGATAAGGCTGTCTTGATAATT GGATTAAATGGTGAATGGGAAACCGAAGGTTATGACAGAGAAAACATGGATT TGCCAAAAAGGACAAATGAATTGGTTCGTGCTGTTTTGAAAGCAAATCCAAAT ACTGTTATCGTTAACCAATCTGGTACCCCAGTCGAGTTCCCTTGGTTAGAAGA CGCAAATGCGCTAGTTCAAGCTTGGTACGGTGGTAATGAATTGGGTAATGCTA TCGCAGACGTCTTATATGGTGACGTGGTTCCAAATGGTAAGTTATCGCTCTCTT GGCCATTTAAGTTACAAGATAATCCAGCCTTTTTAAACTTCAAGACCGAGTTC GGAAGAGTTATTTACGGTGAGGATATATTTGTTGGCTATAGATACTACGAAAA GCTTCAAAGAAAGGTTGCTTTCCCCTTCGGATATGGTCTATCGTATACAACATT CGAACTAGATATTTCTGACTTCAAGGTAACCGATGATAAAATAGCTATTTCAG TTGATGTGAAGAATACTGGTGATAAATTTGCTGGCTCTGAGGTAGTGCAAGTC TACTTCAGTGCTCTAAACTCTAAGGTCTCGAGACCTGTTAAGGAGTTGAAGGG ATTCGAAAAAGTCCATTTGGAACCAGGTGAGAAGAAGACAGTTAATATTGAC CTGGAATTGAAAGACGCAATTTCCTACTTTAACGAAGAGCTCGGTAAATGGCA CGTTGAAGCAGGTGAATACTTGGTTTCAGTTGGTACTTCTTCTGATGATATACT TTCCGTCAAAGAGTTTAAAGTAGAGAAAGAATTGTATTGGAAAGGCTTGTAA).

According to at least one embodiment of the present invention, the polynucleotide sequence encoding the protein that comprises the amino acid sequence of SEQ ID NO: 1 is a polynucleotide encoding the enzyme KmBgl1 of the Saccharomyces Kluyveromyces marxianus; and more specifically, the polynucleotide sequence is a DNA sequence and may be transcribed and translated to generate the enzyme KmBgl1.

According to an embodiment of the present invention, the non-human transformed cell is selected from the group consisting of: a transformed plant cell, a transformed animal cell, a transformed insect cell, transformed Escherichia coli, transformed Bacillus subtilis, transformed Actinomycetes, transformed bacteria, transformed Saccharomyces, and transformed Filamentous bacteria.

According to an embodiment of the present invention, the substrate sesaminol glucosides is selected from the group consisting of: sesaminol 2′-O-β-D-glucopyranoside (sesaminol monoglucoside, SMG), sesaminol 2′-O-β-D-glucopyranosyl(1-2)-O-β-D-glucopyranoside (sesaminol (1-2)diglucoside, SDG (1,2)), sesaminol 2′-O-β-D-glucopyranosyl(1-6)-O-β-D-glucopyranoside (sesaminol (1-6)diglucoside, SDG (1,6)), and sesaminol 2′-O-β-D-glucopyranosyl(1-2)-O-(-β-D-glucopyranosyl(1-2))β-D-glucopyranoside (sesaminol triglucoside, STG).

According to an embodiment of the present invention, the glucosidic bond is selected from the group consisting of: a glucosidic bond bonding between glucose bonded to position 2′ of sesaminol and an aglycone, a β-1,6-glucosidic bond bonding to gentiobiose at position 2′ of sesaminol, a β-1,2 bond bonding to sophoroze at position 2′ of sesaminol, and a β-1,6-glucosidic bond and a β-1,2 bond bondig of branched triglucoside at position 2′ of sesaminol.

The “polynucleotide” described herein refers to DNA or RNA. According to an embodiment of the present invention, the polynucleotide is inserted into an expression vector. Specifically, the polynucleotide of the present invention is preferably introduced into a host after being inserted into an appropriate expression vector. The appropriate expression vector generally includes (1) to (3) as follows: (1) a promoter that may be transcribed in a host cell; (2) a polynucleotide of the present invention that is bonded to the promoter; and (3) an expression cassette with signaling capable of exerting a function in the host cell, as a constituent element, where the function is related to the transcription termination and polyadenylation of an RNA molecule. In regard of a method for preparing the expression vector, plasmids, phages, cosmids or other methods may be used, and the present invention is not limited thereto. In addition, the present invention does not in particular limit the specific type of the vector, and a vector capable of being expressed in the host cell may be appropriately selected. That is, depending on the type of the host cell, an appropriate promoter sequence indeed capable of expressing the polynucleotide of the present invention may be selected and then inserted, together with the polynucleotide of the present invention, into various plasmids to form a vector to serve as the expression vector.

According to a preferred implementation of the present invention, the polynucleotide of the present invention may further include a polynucleotide consisting of a base sequence encoding a secretory signaling peptide; and more preferably, the polynucleotide consisting of the base sequence encoding the secretory signaling peptide is included at a 5′-terminal of the polynucleotide of the present invention.

The expression vector of the present invention depends on the type of a host into which it is to be introduced, and the expression vector includes an expression control region (e.g., a promoter, a terminator, and/or an origin of replication, etc.). In regard of the promoter of the expression vector for bacteria, a conventional promoter (e.g., a trc promoter, a tac promoter, a lac promoter, etc.); in regard of the promoter for Saccharomyces, glyceraldehyde-3-phosphate dehydrogenase promoter, a PH05 promoter or other promoters may be enumerated; and in regard of the promoter for Filamentous bacteria, amlase, trpC or the like may be enumerated. Furthermore, in regard of the promoter for expressing a target gene in a plant cell, a 35S RNA promoter, a rd29A gene promoter, and a recs promoter in a cauliflower mosaic virus may be enumerated; and the enhancer sequence of the 35S RNA promoter of the foregoing cauliflower mosaic virus is added to mannopine sourced from Agrobacterium to synthesize a mac-1 promoter at a 5′-side of a promoter sequence of the enzyme, and so on. In regard of the promoter for animal cell hosts, viral promoters (e.g., a SV40 initial-stage promoter, a SV40 late-stage promoter, etc.) may be enumerated. In regard of the promoter activated under the induction of an external stimulus, a mouse mammary tumor virus (MMTV) promoter, a tetracycline responsive promoter, a metallothionein promoter, a heat shock protein promoter, etc. may be enumerated.

It is preferred that the expression vector includes at least one selective marker. In regard of this marker, a nutritional requirement marker (ura5 or niaD), a drug-resistant marker (hygromycin or zeocin), a geneticin-resistant gene (G418r), a copper-resistant gene (CUP1), a celenin-resistant gene (fas2m or PDR4), etc. may be utilized.

There is no particular limitation to the method for producing a transformant of the present invention, and the method includes, for example, but not limited to: a method for transformation by introducing the expression vector of the polynucleotide of the present invention into a host. In regard of the cell or living organism serving as an object of transformation, various cells or living organisms known at present may be used in an appropriate way. The cell serving as the object of transformation includes, for example, but not limited to: bacteria such as Escherichia coli, Saccharomyces (Saccharomyces cerevisiae), Schizosaccharomyces pombe, Filamentous bacteria (Aspergillus oryzae), Aspergillus sojae, plant cells, animal (other than human) cells, or the like. Appropriate culture media and conditions for the above-mentioned host cells are well known in the technical field. Furthermore, there is no special limitation to the living organism serving as the object of transformation, and various microorganisms, plants, or animals (other than human) illustrated for the host cell above may be enumerated. The transformant is preferably Filamentous bacteria, Saccharomyces or plants. In regard of the host used in transformation, any host capable of generating the sesaminol glucosides may be used. Sesame and other plants that are originally capable of generating at least one sesaminol glucosides, as well as those having essential genes generated by introducing at least one sesaminol glucosides into the cells or living organisms that are originally incapable of generating the sesaminol glucosides, may be used as hosts.

In regard of the method for transforming the host cell, a generally used well-known method may be used. For example, the transformation may be implemented by methods based on electroporation, particle transportation, spheroplasts, lithium acetate or the like, but not limited thereto. Furthermore, when genes are introduced into plants or tissues or cells from plants, methods based on Agrobacterium, a particle gun, PEG, electroporation or the like may be appropriately selected and used.

The protein of the present invention is expressed in a host cell to thus disrupt the cell to obtain the protein of the present invention. The protein of the present invention acts on the substrate sesaminol glucosides to generate the sesaminol glucosides and/or sesaminol of the present invention.

The “enzyme from a transformed cell” is acceptable as long as it is modified using the transformed cell and includes the protein of the present invention, and there is no special limitation. For example, the enzyme may be the transformed cell itself, the disrupted transformed cell itself, the culture supernatant itself of the transformed cell, and their refined products. Therefore, the present provides a method for manufacturing sesaminol and/or sesaminol glucosides. The method includes the steps of: contacting, in a host cell, an enzyme from a non-human transformed cell with sesaminol glucosides having at least one glucosidic bond, and hydrolyzing the at least one glucosidic bond.

The “contacting” refers to coexistence of the enzyme from the transformed cell of the present invention and the sesaminol glucosides having at least one glucosidic bond in a reaction or culture system. For example, the sesaminol glucosides having at least one glucosidic bond is added to a container holding the enzyme from the transformed cell of the present invention; the enzyme from the transformed cell of the present invention is mixed with the sesaminol glucosides having at least one glucosidic bond; or the enzyme from the transformed cell of the present invention is added to a container holding the sesaminol glucosides having at least one glucosidic bond.

In case of where the transformant is Saccharomyces or Aspergillus, the Saccharomyces or Aspergillus transformed with the polynucleotide of the present invention express more proteins of the present invention compared with a wild type. Further, the expressed proteins react with the sesaminol glucosides generated by the Saccharomyces or Aspergillus to generate sesaminol and/or sesaminol glucosides in the cells of the Saccharomyces or Aspergillus or a culture solution, preferably in a culture solution.

In case of where the transformant is a plant, the plant serving as the object of transformation in the present invention means any of a whole plant, a plant organ (e.g., a leaf, a flower, a stem, a root, a seed, etc.), a plant tissue (e.g., a bark, a phloem, a parenchima, a xylem, a vascular bundle, a palisade tissue, a spongy tissue, etc.) or a plant culture cell, or a plant cell in various forms (e.g., a suspension culture cell), a protoplast, a leaf slice, a calluse or the like. In regard of the plant for transformation, the plant may belong to either Monocotyledoneae or Dicotyledoneae. For verification of whether the polynucleotide of the present invention is introduced into the plant, it may be conducted following a method such as PCR, Southern crossing, and Northern crossing. Once the transformed plant with the polynucleotide of the present invention inserted into a genome, the offsprings of the plant may be derived by sexual or asexual reproduction. Furthermore, for example, seeds, fruit, cut ears, tubers, tuberous roots, plants, calluses, protoplasts or the like are obtained from this plant or its offsprings or such a pure line, and based on this, the plant may be produced massively. The plant transformed with the polynucleotide of the present invention (hereinafter referred to as “the plant of the present invention”) contains more proteins of the present invention as compared to a wild type. Based on this, the protein of the present invention reacts with the sesaminol glucosides generated by the plant to thus generate sesaminol in the plant. However, the environment in the plant is not the best for hydrolysis, which additionally inhibits the hydrolysis of the glucosidic bond of the sesaminol glucosides, resulting in sesaminol glucosides remaining the same with the glucosidic bond uncut, or sesaminol glucosides with the glucosidic bond partially cut.

Compared with the wild type, the transformant or its culture solution in several embodiments of the present invention has a higher content of sesaminol and/or sesaminol glucosides; and the extract of the transformant or its culture solution contains a high concentration of sesaminol and/or sesaminol glucosides. The extract of the transformant of the present invention may be prepared by disrupting the transformant by means of glass beads, a homogenizer, or an ultrasonic instrument, centrifuging the disrupted transformant, and collecting the resulting supernatant. In case of where the sesaminol and/or sesaminol glucosides of the present invention being accumulated in a culture solution, the transformant is separated from a culture supernatant by means of a conventional method (e.g., centrifuging, filtration, etc.) at the end of the culture, to obtain the culture supernatant containing the sesaminol and/or sesaminol glucosides of the present invention.

### Embodiments

The present invention will be described below in further detail in detailed description and embodiments. However, it should be understood that these embodiments are intended only to facilitate an easier understanding of the present invention, rather than limiting the scope of the present invention.

### A. Experimental Methods

### Extraction and Analysis of Sesame Pressed Meal Samples

Processing of samples: Sesame pressed meals were ground in a pulverizer and sieved through a 20-mesh sieve; 10 g of the resulting powder was added to 100 mL of n-hexane and stirred overnight at room temperature; the filtrate containing residual oil was removed by suction filtration to retain solids; and the solids were dried in an oven at 40° C. to obtain degreased by-product powder.

Analysis method: 1 g of the degreased sesame pressed meal was weighed, added to 20 mL of 60% methanol, extracted for 1 h by ultrasonic shaking, and centrifuged (9000 rpm, 10 min) to collect the supernatant; the precipitate was then added to 20 mL of 60% methanol, extracted for 1 h by ultrasonic shaking, and centrifuged (9000 rpm, 10 min) to collect the supernatant; and the supernatants were combined, fixed to the volume of 50 mL in a fixed-volume flask, appropriately diluted and then analyzed by HPLC.

### Preparation of Sesame Pressed Meal Extract

1. 60% (v/v) Methanol Extract

150 g of degreased sesame pressed meal was added to 10 times the volume of 60% methanol, stirred overnight at room temperature for extraction, and filtered by suction to collect the filtrate, which was concentrated under reduced pressure to remove the organic solvent, and then freeze-dried into powder.

### 2. Hot-Water Extract

150 g of sesame pressed meal was added to 10 times the volume of water, extracted for 1 h at 121° C., cooled, and centrifuged (9000 rpm, 10 min) to collect the supernatant, which was freeze-dried into powder.

3. 50% (v/v) and 60% (v/v) Ethanol Extracts

150 g of degreased sesame pressed meal was added to 10 times the volume of 50% methanol and 10 times the volume of 60% methanol, respectively, stirred overnight at room temperature for extraction, and filtered by suction to collect the filtrate; the filtrate was concentrated under reduced pressure to remove the organic solvent, and dried overnight in a vacuum oven at 40° C. to obtain the extract; the extract was weighed to calculate the solid content.

### Hydrolysis of Enzyme

1 g of 60% methanol extract was suspended in 3 mL of 50 mM citrate phosphate buffer of pH 5.5 (with the STG content of 1.6 mg/mL), followed by the addition of 114 U (4 mg/mL) Kmbgl1 for reaction for 30 min at 45° C.; an equal volume of methanol was added to stop the reaction; the reaction mixture was centrifuged (1500 rpm, 10 min) to collect the supernatant, which was appropriately diluted and analyzed by HPLC.

### Expression and Separation and Purification of Kmbgl1 Enzyme

### 1. Strain Activation and Seed Bacteria Culture

Kmbgl1::pET21a (+)/E. coli was cryostored (at −80° C.); the strains were seeded to a Lysogeny broth (LB) plate solid culture medium containing 100 μg/mL ampicillin, and cultured for 16 h at 37° C.; and single colonies were picked, seeded into 100 mL of LB liquid culture medium containing 100 μg/mL ampicillin, and cultured for 16 h at 160 rpm at 37° C. to serve as seed bacteria.

### 2. Massive Expression of Recombinant Protein Genes Under Induction of IPTG

5 mL of the seed bacteria were seeded into 500 mL of TB liquid culture medium containing 100 μg/mL ampicillin, and cultured at 150 rpm at 37° C. to OD₆₀₀=0.6, followed by the addition of 200 μL of 500 mM IPTG; the recombinant protein was induced for 24 h at 110 rpm at 16° C. for expression; and the bacterial cells were centrifuged (6000 rpm, 10 min, 4° C.) to collect bacterial cell precipitates, which were stored at −20° C.

The bacterial cells in 500 mL of bacterial solution were resuspended in 50 mL of cell lysis buffer, and shaken until absence of bacterial clots in the liquid, followed by addition of 250 μL of 200 mM protease inhibitor (PMSF); the bacterial cells were disrupted ultrasonically for 30 min (disruption for 8 sec and rest for 4 sec in cycle), and centrifuged at low temperature (15000 rpm, 4° C., 30 min) to remove cell debris and then collect the supernatant, which was the crude extract of enzyme liquid; and the crude extract of enzyme liquid was purified, quick-frozen by liquid nitrogen and stored at −80° C.

### Affinity Analysis of Kmbgl1

The Kmbgl1 sequence was input into the BLAST software for affinity alignment analysis, with the results shown in Table 1 below. The inventor of the present application further conducted enzyme hydrolysis on the species in these analysis results, to test the hydrolysis ability of these species (no shown). The results show that Kluyveromyces lactis, Lachancea fermentati, Saccharomyces mikatae IFO 1815, Brettanomyces anomalus, and Clavispora sp. NRRL Y-50464 each have more than 55% sequence identity to the enzyme of the present invention, where Kluyveromyces lactis and Lachancea fermentati have the activity of hydrolyzing the glucosidic bond of sesaminol and are capable of generating the enzyme of sesaminol, and Saccharomyces mikatae IFO 1815, Brettanomyces anomalus, and Clavispora sp. NRRL Y-50464 fail to completely hydrolyze the sesaminol glucosides into sesaminol, with the enzymes of STG and SDG being mainly present after reaction.

TABLE 1

| Item | | | | Sequence |
|---|---|---|---|---|
| number | Sequence number | Species | Length | Identity |
| Standard | XP_022675159.1 | Kluyveromyces marxianus | 845 | 100 |
| | | DMKU3-1042 | | |
| 1 | QGN15027.1 | Kluyveromyces marxianus | 845 | 99.76 |
| 2 | KAG0671840.1 | Kluyveromyces marxianus | 845 | 99.17 |
| 3 | 3ABZ A | Kluyveromyces marxianus | 845 | 98.82 |
| 4 | P07337.1 | Kluyveromyces marxianus | 845 | 97.87 |
| 5 | QEU61608.1 | Kluyveromyces lactis | 845 | 76.09 |
| 6 | QEU61154.1 | Kluyveromyces lactis | 845 | 74.91 |
| 7 | CDO93564.1 | Kluyveromyces dobzhanskii | 845 | 75.15 |
| | | CBS 2104 | | |
| 8 | XP_454609.1 | Kluyveromyces lactis | 845 | 76.09 |
| 9 | ACF93471.1 | Schwanniomyces etchellsii | 847 | 74.32 |
| 10 | QEU60335.1 | Kluyveromyces lactis | 847 | 74.26 |
| 11 | XP_453086.1 | Kluyveromyces lactis | 765 | 75.93 |
| 12 | SCW01691.1 | Lachancea fermentati | 847 | 65.37 |
| 13 | XP_056077992.1 | Saccharomyces mikatae IFO | 845 | 61.14 |
| | | 1815 | | |
| 14 | XP_038778187.1 | Brettanomyces nanus | 840 | 61.72 |
| 15 | XP_038778857.1 | Brettanomyces nanus | 840 | 61.7 |
| 16 | XP_038778852.1 | Brettanomyces nanus | 832 | 61.14 |
| 17 | XP_455079.1 | Kluyveromyces lactis | 630 | 76.83 |
| 18 | XP_038778601.1 | Brettanomyces nanus | 840 | 59.84 |
| 19 | XP_041135335.1 | Brettanomyces bruxellensis | 841 | 59.5 |
| 20 | AKS48904.1 | Brettanomyces anomalus | 841 | 59.79 |
| 21 | EIF45415.1 | Brettanomyces bruxellensis | 841 | 59.38 |
| | | AWRI1499 | | |
| 22 | KAF6007837.1 | Brettanomyces bruxellensis | 841 | 59.38 |
| 23 | XP_045935579.1 | Saccharomycodes ludwigii | 856 | 59.35 |
| 24 | XP_038777084.1 | Brettanomyces nanus | 841 | 57.07 |
| 25 | KAF6009526.1 | Brettanomyces bruxellensis | 809 | 59.31 |
| 26 | OEJ83753.1 | Hanseniaspora osmophila | 849 | 57.56 |
| 27 | CAH6721386.1 | Candida jaroonii | 838 | 59.37 |
| 28 | OEJ83752.1 | Hanseniaspora osmophila | 849 | 57.56 |
| 29 | CEP22987.1 | Cyberlindnera jadinii | 839 | 56.96 |
| 30 | OEJ84383.1 | Hanseniaspora osmophila | 840 | 58.15 |
| 31 | CDR47499.1 | Cyberlindnera fabianii | 839 | 56.86 |
| 32 | XP_020071577.1 | Cyberlindnera jadinii | 839 | 56.84 |
| | | NRRLY-1542 | | |
| 33 | OEJ87889.1 | Hanseniaspora opuntiae | 846 | 56.88 |
| 34 | GMM42796.1 | Hanseniaspora uvarum | 846 | 56.88 |
| 35 | XP_038780786.1 | Brettanomyces nanus | 843 | 55.49 |
| 36 | XP_002618661.1 | Clavispora lusitaniae | 837 | 56.98 |
| | | ATCC 42720 | | |
| 37 | OVF10368.1 | Clavispora lusitaniae | 837 | 56.98 |
| 38 | KKA02182.1 | Hanseniaspora uvarum | 846 | 56.76 |
| | | DSM 2768 | | |
| 39 | SGZ40447.1 | Hanseniaspora guilliermondii | 846 | 56.99 |
| 40 | CDR44766.1 | Cyberlindnera fabianii | 839 | 56.49 |
| 41 | VEU22172.1 | Brettanomyces naardenensis | 884 | 55.59 |
| 42 | KAF0268396.1 | Hanseniaspora uvarum | 846 | 56.76 |
| 43 | AMK05628.1 | Clavispora sp. NRRL Y-50464 | 837 | 56.86 |
| 44 | XP_001823113.1 | Aspergillus oryzae RIB40 | 839 | 48.60 |

Further, sequence identity alignment analysis was conducted on the above species having the hydrolysis ability, with the results shown in Table 2 below.

TABLE 2

| | Aobg | Clavbg | Hobg | Babg | Smbg | Lfbg | Kmbg | Klbg |
|---|---|---|---|---|---|---|---|---|
| Aobg | 100.00 | 51.69 | 44.15 | 46.76 | 49.39 | 50.36 | 49.52 | 49.27 |
| Clavbg | — | 100.00 | 55.22 | 56.19 | 53.85 | 57.91 | 57.45 | 58.29 |
| Hobg | — | — | 100.00 | 59.50 | 57.01 | 60.10 | 58.18 | 60.45 |
| Babg | — | — | — | 100.00 | 58.61 | 64.52 | 59.74 | 60.81 |
| Smbg | — | — | — | — | 100.00 | 64.38 | 60.97 | 62.38 |
| Lfbg | — | — | — | — | — | 100.00 | 65.33 | 66.63 |
| Kmbg | — | — | — | — | — | — | 100.00 | 76.09 |
| Klbg | — | — | — | — | — | — | — | 100.00 |

*Aobg: Aspergillus oryzae RIB40; Clavbg: Clavispora sp. NRRL Y-50464; Hobg: Hanseniaspora osmophila; Babg: Brettanomyces anomalus; Smbg: Saccharomyces mikatae IFO 1815; Lfbg: Lachancea fermentati; Kmbg: Kluyveromyces marxianus DMKU3-1042; Klbg: Kluyveromyces lactis.

### Determination of Activity of Crude Extract of Enzyme Liquid

The activity was determined by hydrolyzing the glucosidic bond of colorless p-nitrophenyl β-D-glucopyranoside (PNPG) to generate a yellow product p-nitrophenol (PNP), which was then tested for the characteristic of absorbance at 405 nm. The activity was determined before the experiment of each batch of enzymes. With 110 μL of citrate phosphate buffer (50 mM, pH 6.5) and 5 μL of PNPG as the reaction substrate, the enzyme was diluted until the protein concentration was between 6 ppm and 22 ppm; 5 μL of the enzyme to be tested was added to allow for reaction for 10 min in a dry bath at 55° C.; and 120 μL of 0.5 M sodium carbonate was added to stop the reaction and provide an alkaline condition for color development. The absorbance at 405 nm was measured and put into the calibration curve of PNP to obtain the PNP concentration (C_{PNP}) in 240 μL of reaction mixture. A unit (U) of enzyme activity was defined as the amount of enzyme required for generating 1 M PNP product per minute, and the activity concentration (U/mL) of the enzyme solution was calculated according to the equation below:

$$\frac{U}{mL} = \frac{240 μL ( reaction volume ) \times {C}_{PNP} ( M )}{10 min ( reaction time ) \times \frac{5 μL}{1000} mL ( enzyme ) \times dilution factor}$$

### RP-HPLC-UV Analysis

### Analysis Conditions:

Reverse chromatographic column: C18 column YMC-Pack ODS-AM (250×4.6 mm, 5 C)
Mobile phase: water phase: 0.1% (v/v) aqueous formic acid solution;
organic phase: acetonitrile (ACN)
Flow rate: 1.0 mL/min
Injection volume: 20 mL·Detection wavelength: 290 nm
Elution gradient

| Time | % Organic | % Water phase (0.1% |
|---|---|---|
| (min) | phase (ACN) | formic acid (_{aq})) |
| 0 | 15 | 85 |
| 5 | 15 | 85 |
| 30 | 90 | 10 |
| 37 | 15 | 85 |
| 42 | 15 | 85 |

### B. Experimental Results

### Analysis of Contents of Sesaminol-Related Derivatives in Sesame Pressed Meal

### Extraction Effects of Different Solvents on Sesaminol Glucosides

Here, the difference in effects between 60% methanol and 50-60% ethanol on preparation of sesaminol glucosides are discussed, and a method for preparing a matrix subsequently is established.

The results were shown in Table 3 below, in which some extracting solvents were used for extraction at a ratio of 10:1 (v/w), and examples based on water-based solvents were implemented by autoclaved sterilization at 121° C. for 60 min. From Table 3, it can be seen that 60% methanol shows the best extracting effect and can increase the content of sesaminol glucosides in the starting materials to 10.5%, and the extraction with 60% methanol is considered as having 100% recovery rate. There was no significant difference in extraction rates between 50% and 60% ethanol, and their recovery rates were only about 86% compared with extraction with 60% methanol. Extraction by autoclaved sterilization achieved the highest extraction amount but with a relatively higher content of impurities, and only 5.2 g of sesaminol glucosides was contained per 100 g of extract. Moreover, during extraction, oil in the sesame pressed meal was mixed with water to result in emulsification and very turbid filtrate, making it difficult to separate the filtrate from residue by suction filtration, such that the solid and the liquid required to be centrifuged at high speed to break the emulsified phase. Regarding the extraction efficiency, 60% methanol are substantially used in the following embodiments to prepare the sesame pressed meal extract, which is used as the reaction substrate for the subsequent enzyme hydrolysis.

TABLE 3

| Extracting | Yield | SGs content | Recovery |
|---|---|---|---|
| solvent | (%, w/w) | (g/100 g) | rate (%) |
| 60% methanol | 9.5 ± 0.5 | 10.5 ± 0.5 | 100.0 |
| Water | 16.0 ± 0.7 | 5.2 ± 0.6 | 89.6 |
| 50% ethanol | 11.5 ± 0.1 | 7.5 ± 0.1 | 86.3 |
| 60% ethanol | 10.9 ± 0.5 | 7.9 ± 0.3 | 86.6 |

Expression of Kmbgl1 with E. coli Recombinant Protein

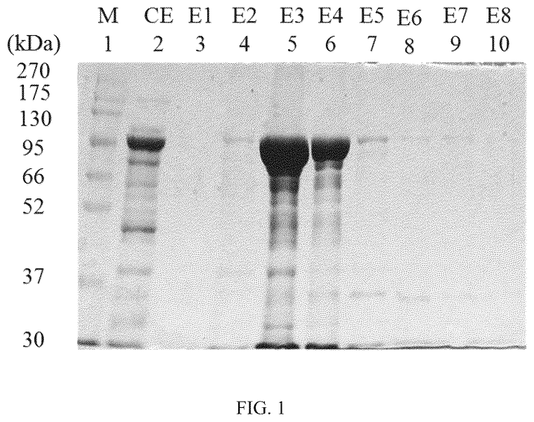

The culture was conducted in a mode of culturing E. coli; 0.5 mM IPTG was added to induce the expression of β-glucosidase at low temperature, E. coli cells were then disrupted to collect intracellular proteins, which were purified by Ni-NTA columns and competed against His6-tag on the target proteins for the coordinate bond by using high-concentration imidazole. The initially purified products were collected in different tubes, and the expression and purification results were analyzed by SDS-PAGE, as shown in FIG. 1. When the concentration of the eluent imidazole was 200 mM, the target proteins were eluted and mainly concentrated in the third and fourth tubes, and a significant band was shown at position 95 kD, which was the enzyme Kmbgl1. The crude extract of enzyme liquid and the partially purified enzyme solution was subjected to Bradford assay for protein quantification, and the activity was then determined by taking pNP-β-Glu as a substrate. The protein in the crude extract of enzyme liquid had a content of 0.85 mg/mL, with the activity of 60.42 U/mg; and the protein in the purified enzyme solution had a content of 0.5 mg/mL, with the activity of 92.14 U/mg. Accordingly, it was determined that all the cultured enzymes had the activity. Afterwards, the crude extract of enzyme liquid of Kmbgl1 having the activity was involved in the reactions in other embodiments.

### Transformation of Kmbgl1 Protein in Extract of Sesame Pressed Meal

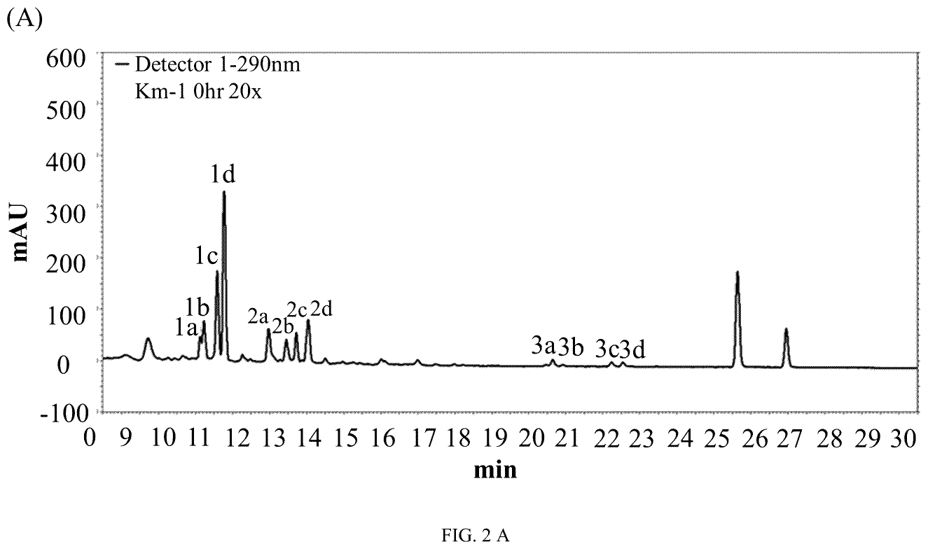

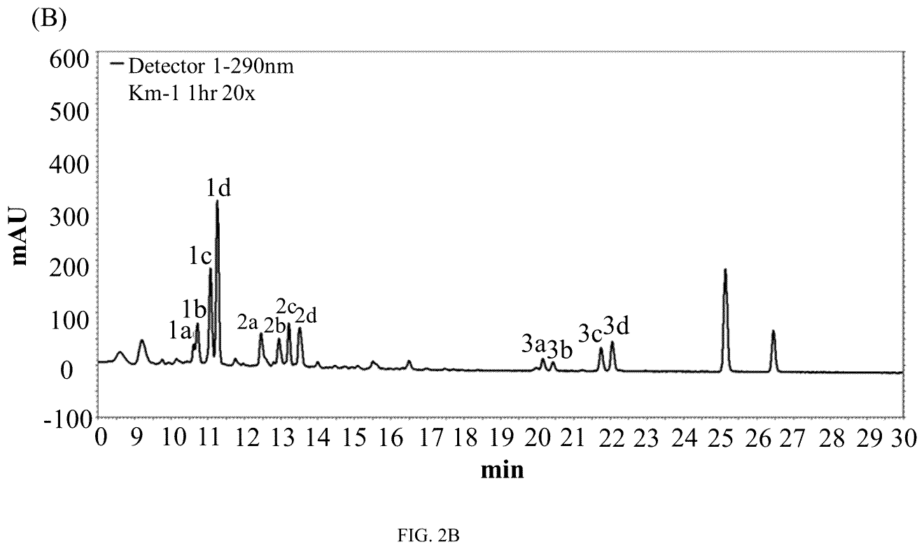

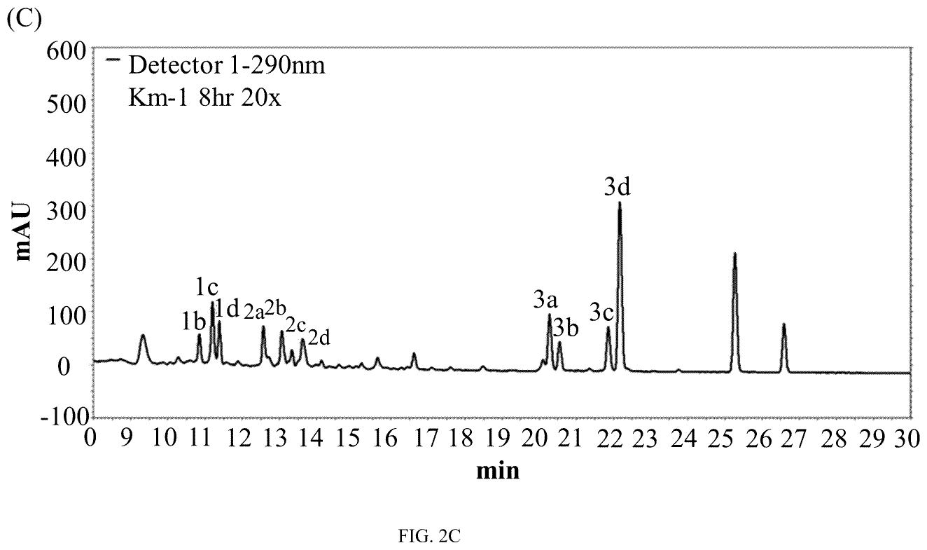

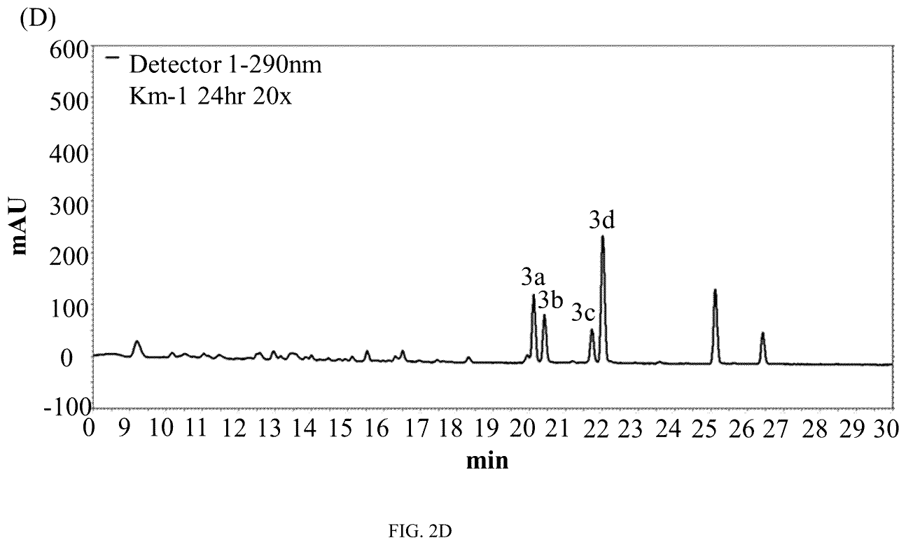

Here, the Kmbgl1 catalyzes the transformation of sesaminol glucosides into sesaminol and Kmbgl1's ability to hydrolyze STG are discussed. In particular, 100 U enzyme was added to 1 g of hot-water extract of sesame pressed meal as the reaction substrate for reaction. The results were shown in FIGS. 2A-2D, in which by reacting at 40° C., Kmbgl1 gradually hydrolyzed the sesaminol glucosides in the sesame pressed meal extract into sesaminol; and during the reaction, the glucosidic bonds on STG and SDG were directly cut off for transformation into sesaminol.

### Test of Hydrolysis Conditions of Kmbgl1 Enzyme

From the above experiments, the reaction substrate and the extraction mode, as well as the enzymes used for catalysis and the like, were approximately determined; and the following, tests were further conducted with respect to different reactions so as to identify the most optical conditions for catalytic hydrolysis by Kmbgl1.

### Effects of the Most Optical Amount of the Enzyme Added on the Generation of Sesaminol Glucosides

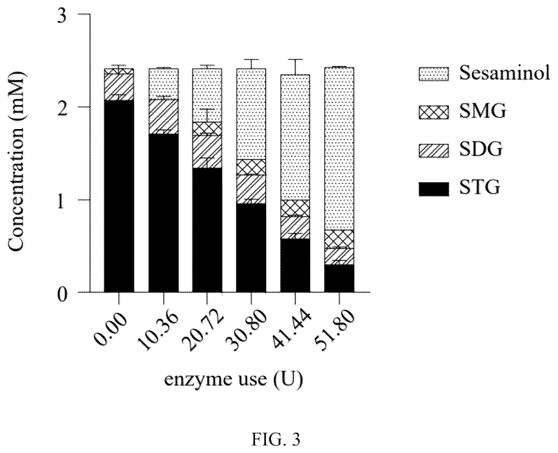

First, the present invention will discuss the most optical amount of the enzyme added; and thus, the extract rich in sesaminol glucosides was selected as the reaction substrate. The results were shown in FIG. 3, as the amount of enzyme added increased, the production amount of sesaminol also gradually increased; and when the amount of the enzyme added reached to 51.8 U, the conversion rate of sesaminol reached to 80% based on quantitative calculation.

### Effects of the Most Optical Reaction Time on the Generation of Sesaminol Glucosides

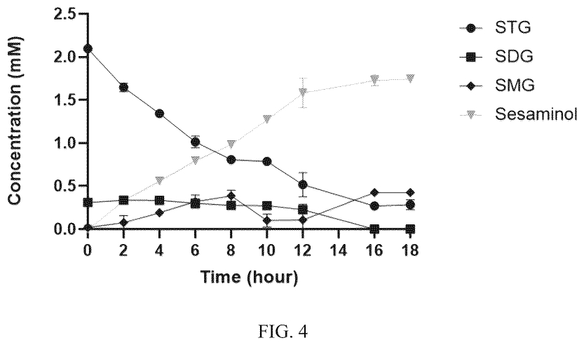

Based on the results of the preceding test, the subsequent experiment in this section was conducted with 0.1 g as the amount of substrate added and 51.8 U as the most proper amount of enzyme, and the most proper reaction time was further discussed. The results were shown in FIG. 4, in which STG and SDG in the crude extract gradually decreased as the increase of the reaction time, and were then transformed into sesaminol. The enzyme continuously exerted an action under the reaction condition of 37° C. until the reaction lasted for 16-18 hours, in which there was no significant generation of sesaminol. Accordingly, the most proper reaction time for the enzyme of the present invention was 16 hours, after which the activity of the enzyme might be lost. Therefore, the reaction time used in all the subsequent tests was 16 hours.

### Effects of the Most Proper Reaction pH on the Generation of Sesaminol Glucosides

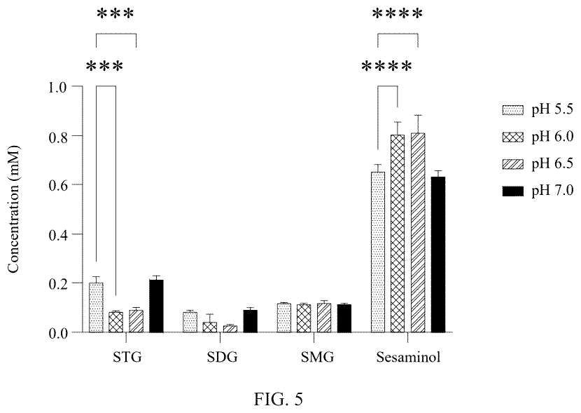

pH value was also an important factor affecting the hydrolysis of enzymes, and thus, the most proper pH value for the reaction of sesaminol glucosides was then studied in this test. According to the literature, it was pointed out that Kmbgl1 was more stable at pH 6.0-9.0, and showed better reactivity under weakly acidic condition. Therefore, pH 5.5, 6.0, 6.5 and 7.0 were selected in this test for the subsequent experiments. The results were shown in FIG. 5, in which the generation amount of sesaminol was higher in condition of pH 6.0 and 6.5, indicating that the optimal pH value for Kmbgl1 and sesaminol glucosides should be between 6.0 and 6.5.

### Effects of the Most Proper Reaction Temperature on the Generation of Sesaminol Glucosides

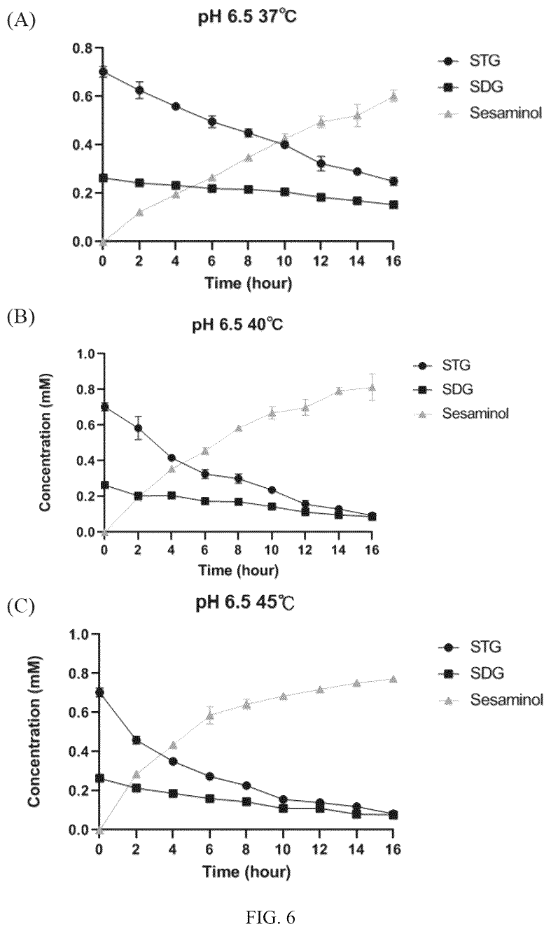

After the verification of the optimal pH values for the hydrolysis of sesaminol glucosides by Kmbgl1, considering that Kmbgl1 was relatively stable at pH 6.5, the effects of different temperatures on the transformation of sesaminol glucosides were determined at pH 6.5 in this test. The results were shown in FIG. 6, in which the highest generation rate of sesaminol was up to 80% after reaction for 16 h at 40° C. with the same amounts of substrate and enzyme added and the same reaction pH. During the continuous reaction of the enzyme at 37° C., the average amount of the sesaminol generated at each time point was about the same, but the total sesaminol transformation rate was the lowest among the three temperatures, indicating that the activity of enzyme was relatively low at this temperature. After 2 hours of reaction at 45° C., the amount of the sesaminol generated was higher than those in another two cases, but after 12 hours of reaction, the amount of the sesaminol generated gradually decreased and even does not generate, indicating that the increase in temperature did increase the reaction rate, but shortened the reaction time at the same time. Regardingly, 40° C. was considered the optimal reaction temperature based on the final total transformation rate.

### Hydrolysis Test of Different Enzymes

Here, the differences of enzymes having different identities in preparation of sesaminol glucosides are discussed. Specifically, the crude extracts of enzyme liquid from different Saccharomyces sources were used in this test to hydrolyze the hot-water extract (HWE) of sesame pressed meal, and the content of the sesaminol glucosides generated therefrom was further analyzed. The experiment was briefly described as follows: 0.5 g of HWE powder was weighed and added to a test tube with a cap; the citrate phosphate buffer (pH 6.5, 50 mM) containing 100 activity units (U) of the enzyme was added; after full mixing, a sample was collected at 0 h; then, the test tube was placed in a shaking water bath at 40° C. and shaken back and forth at 100 rpm to react for 16 h, and another sample was collected; and during sample collection, the reaction was stopped with a doubled volume of methanol, the reaction mixture was diluted with 40 times methanol and centrifuged to remove precipitates, and the supernatant was collected for RP-HPLC-UV analysis.

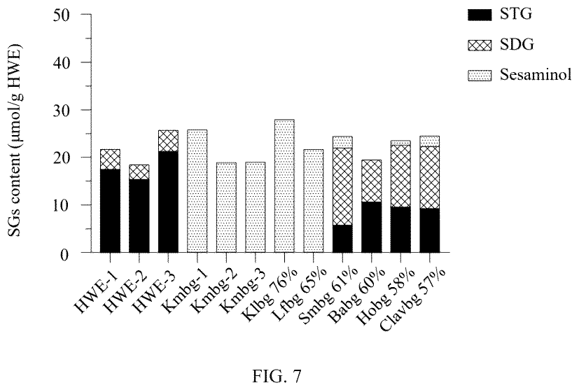

The results were shown in FIG. 7, in which the enzymes Kmbgl1 of the present invention were capable of effectively transforming the sesame pressed meal into the sesaminol glucosides, and K1bg (SEQ ID NO: 3, MSNFDIEQTLSELTRDEKISLLSAVDFWHTKEIERLGIPSVRVSDGPNGIRGTRFFDS VPSGCFPNGTGLASTFDDELLKEAGKLMAKEAVAKNAAVILGPTTNMQRGPLGG RGFESFSEDPYLAGVATSSVVQGMQSEGIAATVKHFVCNDLEDQRFASNSILSERA LREIYLEPFRLAIKNADPVCLMTAYNKVNGEHCSQNKKLLLDILRKEWNWDGMI MSDWYGTYTTAASIKNGLDIEFPGPTRWRTNELVSHSLNSKEQISIYDVDDRVRQ VLKMIKFVVDNQEKTGIVQNGPETTSNNTKETSELLRKIAADSIVLLKNENSILPLK KEESIVVIGPNAKAKASSGGGSASVNSYYVISPYEGIVKKVGKEVPYTIGAESHKT LSNLIEQLVVDPSKPAEGDNAGATGSFYSEPVEKRAKDESPFHVATFKHSFNLLFD FKHEKIDTTNPIFYITLEGYFTPEEDADYIFGLQVFGTGVLYLDDELLIDQRKGQVS GDFCFGAGTIEKTKTVTLQKGKAYKVRIEYGSGPTSELVSEFGSGALQVGVTKAI DADEEIKKAAKLAAAHDKAILCIGLNAEWESEGHDREDMTLPARTNDLVRAVLE ANPNTVIVNQSGTPVEFPWLQKANALVQAWYGGNELGNAIADVLYGDVVPNGK LSLSWPLKLEDNPAYLNFKTEFGRVVYGEDIFIGYRFYEKLQKRVAFPFGYGLSYT EFALSNLQVQINDEVISVSVDVKNTGEKYAGSEVVQVYIAATESSVSRAVKELKG FKKVLLQPGQTETAKIDLVLKDSVSFFDEEVGKWCSEAGQYKVLVGTSSDDIVLS ESFDVEKTSYWSGL) and Lfbg (SEQ ID NO: 4, MSKFDIEELIGELTLQEKIALIAAKDFWHTSPVERLGIPSVRVSDGPNGVRGTKFFN SVPSAAFPNGTGLASTFDTELLEEAGQLMAVEAAHKNASVILGPTTNIARGPLGG RGFESFSEDPYLSGMCTAALVNGMQSRGIAATVKHYVCNDLEDQRFSSNSIVTER ALREIYLEPFRLAVKYANPVCIMSSYNKVNGTHCSQSKKLLDDILREEWGWDGMI TSDWFGTYSSADAIKNGLDIEFPGPTKWRKSELISHLISSKEGISEEDVNTRVRNVL KMIKFAVDNKDKTGIIENGPESDANNTPETAAKLRKIAADSIVLLKNENNVLPLSK DESIVVIGPNAKTKFMSGGGSASLNPYYVVPIYDGIKSKLGKDPEYSIGCTSNKTL NGLWEACVIDPSKGNQADNIGAQAIFYTKPVEDRSPDEKPIDSTTIKQSYLTLFDY KNPAVEESNPLFYVDFEGYYTPEEDGDYEIGLQVYGTALLFIDGELVVDNKTKQT KGTFCFSAGTIEEKAIVSMKAGKSYKFRVEYGSGPTSQTASDFGAGGMQVGIAKK IDENEEIAHAAQLAKEHDKVVLCIGLNGEWESEGYDRENMTLPKKTNDLVRAVL RANPNTVVVNQSGTPVEMPWISECNALLQCWYGGNELGNAVADIAFADVVPSA KLSLSWPFKNEDNPAYLNFATESGRVLYGEDVFVGYRFYEKLQRQVAFPFGYGL SYTTFTFENLEVTADESKEILSVQLDVTNSGSKYAGAEVVQVYVAPTKSGITRPVK ELKGFKKVYLEPNETKKVSLELPLKDSISYFEEYHNKWCAEAGEYQLLAGSSSDD TQLISSFELSKTFYWKGL), which had 76% and 65% sequence identities to the Kmbgl1 sequence respectively, shared the same hydrolysis ability, and were capable of hydrolyzing the sesame pressed meal into the sesaminol glucosides.

In another aspect, Smbg (SEQ ID NO: 5, MTFDIEKVLSELTTNEKISLIAAEDFWHTTPIKRLDIPSVRVSDGPNGIRGTKFFNSV PSAAFPNGTALASTFDKELLKEVGARMADEAIQKNAGVILGPTINIQRGPLGGRGF ESFSEVPYLSGIAASCIVNGIQSRGVAATLKHFVCNDLEDQRMSSNSIVTCRALREI YLEPFKLAVKYSDPQCIMTSYNKVNGVHCSNSKNLLIDILRDEWKWGGMVMSD WFGTYSVDSIKNGLDIEFPGPSKWRSLDLLKSNLDSKAGITISNIDDCVRHVLKLV HYVSENSKKTQIKDHGPETTLNNTEHMSKHLRKVASESIVLLKNVDDILPLKKESS VVVIGPNAKAKSYSGGGSASLQPYYVITPYEGICEKIGRNVEYTAGCDSRKTLSGL IEAMVVDPCQPAEGDNIGIIAQYFMDPANQRSADVEPFDTHRVTQSYVTLFDYTH PNIDPIMPFFYIHFEGFFTPEEDGEYIFGVQVFGTALFYIDDKLEIDNKTHQTKGSFC FGAGTREETCEKYLVKGHQYRIRIEYGSGPTSSVAADFGYGGIQVGFAKKLNADE EIARAVQLAKTNDNVILCIGLNGEWESEGYDRENMSLPKNNDRLISAVLTANPNTI VVNQSGMPVELPWVDQCKALLQCWYGGNELGNAIADVLYGDVVPSGKLSISWP YMCHDNP AFLNFKTESGRVLYGEDIYVGYRFYEKVRRQVAFPFGHGLSYTTFRFD DLIVSIDETLDLLETSLNVTNTGDTIAGKEVIQVYVSHNESTIGRPIKELKGFQKVFL KPNETKKVTMKLSLKDSISYFDEEKQLWCATEGIYQILVGSSSKDIKLTERFDVNK TSYWKGL), Hobg (SEQ ID NO: 6, MPIDFNVDRLLTELTLDEKLSLLAGQDWWHTAAIERLNIPSVRVSDGPNGIRGTRF FACVPSACFPNGTALASTFNEEILESAGELMALEAKHKGAKVILGPTANIQRGPLG GRGFESFSEDPYLSGVATAAVVNGIQKSNEIAATVKHFVCNDMEHERFSSNSIVSE RALREIYLEPFRLAVKHAQPKLFMTSYNKLNGIHCSSSKKLLQDILRNDWNSGAT VISDWFGITDIVDSIQNGLDIEFPGPTRYRKPEILKNLLMCKTETREGGQFSIEHIDA RARKVLELVKYFVEAEQSTDFPTNEDDHNNTFETAQFLRRLGNETIVLLKNETKL LPLDKKDDIVVIGPNAKAKNSSGGGCAALNGYYTISPLEGIANVTQRKTGDIPYTK GCDNHKNLSNLIEQCTNDADPEKKGAEMNFYTQPREVRGKEKPFDSYIIDQSFITL FDYKHEKVDEKKRLFYCTIEGYFIPKEDGDFEFQCQVLGTALFYIDDKLVINNKDD QTAGNFGFGSGTAPKNNIVTLEKGRKYKIFVDYGSGVTSKLSQSIAAGALQIGVN KVIDAEAEIKKAAELASKHDKVILVIGLNGEIESEGYDRDNMQLPRRTNDLVTAV LKANPNTVIVNQSGTPVEFPWLQQATTLLQAYYGGNELGNSIADVVFGDANPSG KLSLSWPLKNEDNPAYLNFKTVMGRVLYGEDIYVGYRFYEKLQRQVAYPFGHGL SYTTFKFNELDVSGDDESLKVELSVANTGKVDGKEVVQVYVARTSPSAVPRPVK ELKKFKKVALKAGESAKVELTLSVKDSCSYFDEFHNQWHLEAGKYQVLVGSSSD DIHLIGDFEVKESEFWLGL) and Clavbg (SEQ ID NO: 7, MADIDVEKVLSELTLAEKIGLTAGVDFWHTYKVERLGVPTLRLSDGPNGVRGTK FVNGAPSACFPCGTGLASTFNKDLLYKAGRLMADEAKHKSAHVILGPTTNMQRG PLGGRGFESFSEDPHLAGMASASIVKGMQDNDIAATIKHFVCNDLEHERNSSDAIV TERALREIYLEPFRLAVKYADPKSFMTAYNKVNGEHVSQSHRILEQILREEWNWD GLVMSDWYGAYTAKESLTNGLDLEMPGPSGMRTVQNISHMVNSRELNIKYLDER VRNVLKLVKWCARSKLEERGPETTENNTPETRALLNKIASELVVLLKNNDSVLPL KKEESIAVIGPNAKFAAYCGGGSASLLSYYTTTPYDAIKEKLGHEPKYAVGCYAH QMLPGFSLSPYTKNPVTGKSGVNCKLYNDAPGTKNRRQFDEFDITMSPIILFDYRH PAIVDELFYMDITGDLTPEESGEYEFSLTVSGTAQLFIDDKLVIDNKNSQTLGTAFF GTGTIEMKQKVPLDAGKTYNVRVEFGSSKTSKLRPLSVISFGGCVSIGMCKVIDPK EEIAKAVELAKSVDKVVLLIGLNAEWESEGFDRPDMELPLLTNDLVEAVLAANPN TVVVNQSGTPVEMPWLSKANALVHAWYGGSEAGNAIANVLFGDVNPSGKLSLS WPFKNSDNPAYLNFHTERGRVLYGEDIYIGYRFYDKLQRRVAFPFGYGLSYTTYK YSDLNVTVNEEDDSLTASVTVENTGSKDGAETVQFYVAPKTSEVARPVKELKGF DKVFVKAGEKATAKVQLSLKDSASFFDEYHDKWSLEKGTYEVQVGKSSDDVELI QEFKVKESKLWSGL), which have 61%, 58 and 57% sequence identities respectively, also showed slight hydrolysis ability.

Recovery of Sesaminol from the Reaction Mixture of Enzymes

Due to the poor water solubility of sesaminol, the reaction mixture in this test was held in a low-temperature environment to promote the precipitation of sesaminol. In this embodiment, 60% methanol extract and hot-water extract were selected as the reaction substrate; the reaction occurred with the same amount of enzyme added under the same reaction conditions; the precipitate derived by low-temperature precipitation was used as a sesaminol concentrate; and the feasibility of recovering the sesaminol by precipitation was evaluated. The results were shown in Table 4 below, in which the yield rate was calculated by (the weight of sesaminol concentrate/the weight of starting material)×100%. There was no significant difference in yield rate between the two (11.1% and 10.7%); however, there was a two-fold difference in the content of sesaminol, indicating the influence of the purity of the reaction materials on the purity of the final product. By the low-temperature precipitation, the recovery rates of both were more than 95%, indicating the feasibility of the low-temperature precipitation, and there was substantially no loss of sesaminol during recovery process.

TABLE 4

| Starting | Yield rate | Sesaminol | |
|---|---|---|---|
| material | (%, w/w) | Content (%, w/w) | Recovery Rate (%) |
| 60% methanol | 11.1 | 42.4 | 96.9 |
| extract | | | |
| Hot-water extract | 10.7 | 21.2 | 95.0 |
| (121° C., 1 h) | | | |

Establishment of Method for Preparing Sesaminol from Sesame Pressed Meal Extract

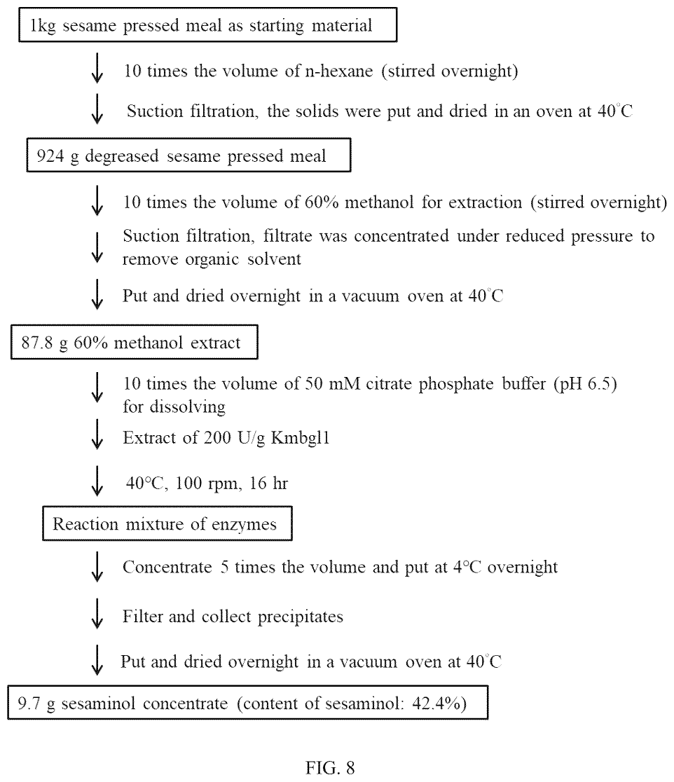

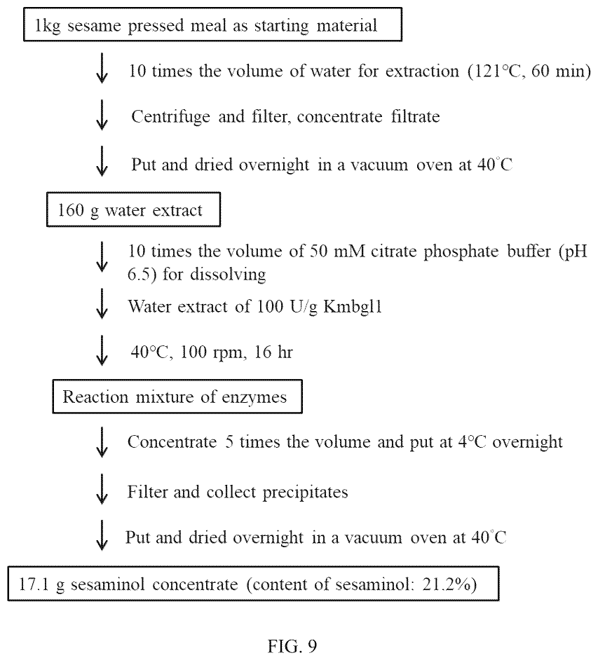

The preceding test determined the feasibility of recovering sesaminol by low-temperature precipitation, and the following, the procedures of preparing sesaminol was studied in practice. These were shown in FIG. 8 and FIG. 9. In a first method (FIG. 8), the sesame pressed meal as the starting material was degreased and extracted with 60% methanol to prepare a sesame pressed meal extract; then, the extract with the amount of 200 U/g enzyme solution was added to allow for reaction for 16 h at 40° C. and pH 6.5; and the sesaminol as the final product had a content of 42.4%, with the total recovery rate of 96.9%. In a second method (FIG. 9), the sesame pressed meal as the starting material was added to 10 times the volume of water, and extracted for 60 min at 121° C. to prepare a water extract; then, the water extract with the amount of 100 U/g enzyme solution was added to allow for reaction for 16 h at 40° C. and pH 6.5, and sesaminol was then recovered by low-temperature precipitation; and the sesaminol as the final product had a content of 21.2%, with the total recovery rate of 85.0%. Accordingly, the first method was ideal, and the second method was more practically feasible.

In summary, the method for generating sesaminol and/or sesaminol glucosides provided by the present invention can shorten the processing time and improve the yield of sesaminol to reduce the manufacture cost, which are in line with the application requirements in industry.

All ranges provided herein are intended to include each specific range within a given range and a combination of subranges within that given range. Furthermore, unless otherwise noted, all ranges provided herein include their end points. Thus, the range of 1-5 specifically includes 1, 2, 3, 4, and 5, as well as subranges such as 2-5, 3-5, 2-3, 2-4, 1-4, etc.

While the present invention has been detailed above, the embodiments described herein are only some preferred ones of the present invention and should not be viewed as restrictive of the scope of the present invention. Any equivalent change or modification based on the appended claims shall fall within the scope of the invention.

What is claimed is:

1. A method for generating sesaminol or sesaminol glucosides, comprising the steps of: reacting a protein with a substrate sesaminol glycoside having at least one glucosidic bond, and catalyzing hydrolysis of at least one glucosidic bond;
wherein the protein is selected from the group consisting of (1) to (3): (1) a protein that comprises an amino acid sequence of SEQ ID NO: 1; (2) a protein that comprises an amino acid sequence derived by deletion, substitution, insertion and/or addition of one or more amino acids in the amino acid sequence of SEQ ID NO: 1 and has an activity of catalyzing the hydrolysis of at least one glucosidic bond; and (3) a protein that comprises an amino acid sequence having more than 60% sequence identity to the amino acid sequence of SEQ ID NO: 1 and has an activity of catalyzing the hydrolysis of at least one glucosidic bond.

2. The method of claim 1, wherein the substrate sesaminol glycoside is selected from the group consisting of: sesaminol 2′-O-β-D-glucopyranoside (sesaminol monoglucoside, SMG), sesaminol 2′-O-β-D-glucopyranosyl(1-2)-O-β-D-glucopyranoside (sesaminol (1-2)diglucoside, SDG (1,2)), sesaminol 2′-O-β-D-glucopyranosyl(1-6)-O-β-D-glucopyranoside (sesaminol (1-6)diglucoside, SDG (1,6)), and sesaminol 2′-O-β-D-glucopyranosyl(1-2)-O-(-β-D-glucopyranosyl(1-2))β-D-glucopyranoside (sesaminol triglucoside, STG).

3. The method of claim 1, wherein the substrate sesaminol glycoside is a 60% (v/v) methanol crude extract.

4. The method of claim 1, wherein the sesaminol or sesaminol glucosides is/are selected from the group consisting of: sesaminol (1-6)diglucoside (SDG (1,6)), sesaminol (1-2)diglucoside (SDG (1,2)), and sesaminol.

5. The method of claim 1, wherein a temperature for reacting the protein with the substrate sesaminol glycoside is 37° C. to 45° C.

6. The method of claim 1, wherein reacting the protein with the substrate sesaminol glycoside occurs at pH 5.5 to pH 6.5.

7. The method of claim 1, wherein a reaction time for the protein with the substrate sesaminol glycoside is 16 hours.

8. The method of claim 1, wherein the glucosidic bond is selected from the group consisting of: a glucosidic bond bonding between glucose bonded to position 2′ of sesaminol and an aglycone, a β-1,6-glucosidic bond bonding to gentiobiose at position 2′ of sesaminol, a β-1,2 bond bonding to sophoroze at position 2′ of sesaminol, and a β-1,6-glucosidic bond and a β-1,2 bond bonding of branched triglucoside at position 2′ of sesaminol.

9. A method for generating sesaminol or sesaminol glucosides, comprising the steps of: in a host cell, an enzyme from a non-human transformed cell is reacted with substrate sesaminol glycoside having at least one glucosidic bond, and catalyzing hydrolysis of at least one glucosidic bond;
wherein a polynucleotide is introduced into the non-human transformed cell, and the polynucleotide is selected from the group consisting of (1) to (5): (1) a polynucleotide encoding a protein that comprises an amino acid sequence of SEQ ID NO: 1; (2) a polynucleotide encoding a protein that comprises an amino acid sequence derived by deletion, substitution, insertion and/or addition of one or more amino acids in the amino acid sequence of SEQ ID NO: 1 and has an activity of catalyzing the hydrolysis of at least one glucosidic bond; (3) a polynucleotide encoding a protein that comprises an amino acid sequence having more than 60% sequence identity to the amino acid sequence of SEQ ID NO: 1 and has an activity of catalyzing the hydrolysis of at least one glucosidic bond; (4) a polynucleotide encoding a protein that has an activity of catalyzing the hydrolysis of at least one glucosidic bond, which hybridizes with a polynucleotide comprising a complementary base sequence at a highly demanding condition, wherein the complementary base sequence is complementary to the polynucleotide encoding the protein comprising the amino acid sequence of SEQ ID NO: 1; and (5) a polynucleotide comprising a base sequence of SEQ ID NO:2.

10. The method of claim 9, wherein the polynucleotide is inserted into an expression vector.

11. The method of claim 9, wherein the non-human transformed cell is selected from the group consisting of: a transformed plant cell, a transformed animal cell, a transformed insect cell, transformed Escherichia coli, transformed Bacillus subtilis, transformed Actinomycetes, transformed bacteria, transformed Saccharomyces, and transformed Filamentous bacteria.

12. The method of claim 9, wherein the substrate sesaminol glucosides is selected from the group consisting of: sesaminol 2′-O-β-D-glucopyranoside (sesaminol monoglucoside, SMG), sesaminol 2′-O-β-D-glucopyranosyl(1-2)-O-β-D-glucopyranoside (sesaminol (1-2)diglucoside, SDG (1,2)), sesaminol 2′-O-β-D-glucopyranosyl(1-6)-O-β-D-glucopyranoside (sesaminol (1-6)diglucoside, SDG (1,6)), and sesaminol 2′-O-β-D-glucopyranosyl(1-2)-O-(-β-D-glucopyranosyl(1-2))β-D-glucopyranoside (sesaminol triglucoside, STG).

13. The method of claim 9, wherein the sesaminol or sesaminol glucosides is/are selected from the group consisting of: sesaminol (1-6)diglucoside (SDG (1,6)), sesaminol (1-2)diglucoside (SDG (1,2)), and sesaminol.

14. The method of claim 9, wherein the glucosidic bond is selected from the group consisting of: a glucosidic bond bonding between glucose bonded to position 2′ of sesaminol and an aglycone, a β-1,6-glucosidic bond bonding to gentiobiose at position 2′ of sesaminol, a β-1,2 bond bonding to sophoroze at position 2′ of sesaminol, and a β-1,6-glucosidic bond and a β-1,2 bond bonding to branched triglucoside at position 2′ of sesaminol.


SEQUENCE LISTING (7 sequences)

SEQ ID NO: 1 - protein, 845 aa, Kluyveromyces marxianus
MSKFDVEQLLSELNQDEKISLLSAVDFWHTKKIERLGIPAVRVSDGPNGIRGTKFFDGVPSGCFPNGTGLASTFDRDLLETAGKLMAKESIAKNAAVILGPTTNMQRGPLGGRGFESFSEDPYLAGMATSSVVKGMQGEGIAATVKHFVCNDLEDQRFSSNSIVSERALREIYLEPFRLAVKHANPVCIMTAYNKVNGEHCSQSKKLLIDILRDEWKWDGMLMSDWFGTYTTAAAIKNGLDIEFPGPTRWRTRALVSHSLNSREQITTEDVDDRVRQVLKMIKFVVDNLEKTGIVENGPESTSNNTKETSDLLRKIAADSIVLLKNKNNILPLKKEDNIIVIGPNAKAKTSSGGGSASMNSYYVVSPYEGIVNKLGKEVDYTVGAYSHKSIGGLAESSLIDAAKPADAENSGLIAKFYSNPVEERSDDEEPFHVTKVNRSNVHLFDFKHEKVDPKNPYFFVTLTGQYVPQEDGDYIFSLQVYGSGLFYLNDELIIDQKHNQERGSFCFGAGTKERTKKLTLKKGQVYNVRVEYGSGPTSGLVGEFGAGGFQAGVIKAIDDDEEIRNAAELAAKHDKAVLIIGLNGEWETEGYDRENMDLPKRTNELVRAVLKANPNTVIVNQSGTPVEFPWLEDANALVQAWYGGNELGNAIADVLYGDVVPNGKLSLSWPFKLQDNPAFLNFKTEFGRVIYGEDIFVGYRYYEKLQRKVAFPFGYGLSYTTFELDISDFKVTDDKIAISVDVKNTGDKFAGSEVVQVYFSALNSKVSRPVKELKGFEKVHLEPGEKKTVNIDLELKDAISYFNEELGKWHVEAGEYLVSVGTSSDDILSVKEFKVEKELYWKGL

SEQ ID NO: 2 - DNA, 2538 bp, Kluyveromyces marxianus
  source 1..2538: mol_type = genomic DNA
atgtctaaatttgatgttgaacagttattgagtgaattgaaccaggatgaaaagatttccttactctctgcagttgatttctggcatactaagaagattgaacggttgggaattccagcggtgagggtttctgatggtccaaatggtattagagggacaaagttctttgatggggttccttcaggatgtttccctaatggtaccgggttggcatctacttttgatcgcgacctgcttgagacagcaggtaagttgatggccaaggaatcgattgcgaagaatgctgctgtgattttgggtccaaccacaaacatgcaacgtggtcctttgggtggtcgtggttttgaatcattttctgaagatccataccttgctggtatggcaacttcttctgttgttaaaggtatgcagggcgaaggtattgctgctaccgttaagcattttgtttgtaacgatttggaagaccaacgtttctcttcaaactcaattgtttctgaaagggctcttagagaaatttacttggagcccttcagattggcagttaaacatgccaatcctgtttgtataatgactgcctataacaaggtcaatggcgaacattgctcccaatccaagaagctattgatcgacattttgagagacgagtggaaatgggacggtatgttaatgtccgactggttcggtacatatacgactgccgcagctatcaagaatgggttggatatcgagtttcctggaccaacaagatggagaacacgtgctttagtgtctcactcactcaactccagagaacaaatcactactgaagatgttgatgatcgtgttagacaagtgctaaaaatgattaagttcgttgttgacaatttagagaaaacaggtattgtggagaatggcccagaatctacttcaaacaacaccaaggaaacctcggacctgttgagaaagattgctgctgactctattgttttattgaagaacaaaaacaatatcttacctctaaagaaagaagacaatatcattgtcattggcccaaatgctaaagcaaagactagttctggtggtggttcagcatccatgaactcctactatgtcgtttctccgtatgaaggtatcgtcaataagctgggcaaagaggtcgactacaccgtaggcgcctattcacacaaatctattggaggtttggccgagagtagtttgatcgatgctgcaaaaccagcagatgctgaaaattctggattaattgccaagttttactccaatccagtagaagagagatctgacgatgaagaaccattccacgttaccaaagtcaatagatccaatgttcacttatttgatttcaaacatgagaaagtggatccaaagaacccttacttttttgtaaccttaaccggacagtacgtgccccaagaagatggtgattatatcttcagtcttcaagtttatggttctggtttgttctacttaaacgatgagttgattattgaccaaaagcacaaccaagaaaggggtagtttctgctttggagctggtaccaaagaaagaaccaaaaagttgactttgaagaagggccaagtttataatgtaagagttgagtacggttctggcccaacttcaggtttggttggggaattcggtgcaggtggattccaagctggtgtcatcaaggccatcgacgatgacgaggagattagaaacgcagcggaattagcagctaagcatgataaggctgtcttgataattggattaaatggtgaatgggaaaccgaaggttatgacagagaaaacatggatttgccaaaaaggacaaatgaattggttcgtgctgttttgaaagcaaatccaaatactgttatcgttaaccaatctggtaccccagtcgagttcccttggttagaagacgcaaatgcgctagttcaagcttggtacggtggtaatgaattgggtaatgctatcgcagacgtcttatatggtgacgtggttccaaatggtaagttatcgctctcttggccatttaagttacaagataatccagcctttttaaacttcaagaccgagttcggaagagttatttacggtgaggatatatttgttggctatagatactacgaaaagcttcaaagaaaggttgctttccccttcggatatggtctatcgtatacaacattcgaactagatatttctgacttcaaggtaaccgatgataaaatagctatttcagttgatgtgaagaatactggtgataaatttgctggctctgaggtagtgcaagtctacttcagtgctctaaactctaaggtctcgagacctgttaaggagttgaagggattcgaaaaagtccatttggaaccaggtgagaagaagacagttaatattgacctggaattgaaagacgcaatttcctactttaacgaagagctcggtaaatggcacgttgaagcaggtgaatacttggtttcagttggtacttcttctgatgatatactttccgtcaaagagtttaaagtagagaaagaattgtattggaaaggcttgtaa

SEQ ID NO: 3 - protein, 845 aa, Kluyveromyces lactis
MSNFDIEQTLSELTRDEKISLLSAVDFWHTKEIERLGIPSVRVSDGPNGIRGTRFFDSVPSGCFPNGTGLASTFDDELLKEAGKLMAKEAVAKNAAVILGPTTNMQRGPLGGRGFESFSEDPYLAGVATSSVVQGMQSEGIAATVKHFVCNDLEDQRFASNSILSERALREIYLEPFRLAIKNADPVCLMTAYNKVNGEHCSQNKKLLLDILRKEWNWDGMIMSDWYGTYTTAASIKNGLDIEFPGPTRWRTNELVSHSLNSKEQISIYDVDDRVRQVLKMIKFVVDNQEKTGIVQNGPETTSNNTKETSELLRKIAADSIVLLKNENSILPLKKEESIVVIGPNAKAKASSGGGSASVNSYYVISPYEGIVKKVGKEVPYTIGAESHKTLSNLIEQLVVDPSKPAEGDNAGATGSFYSEPVEKRAKDESPFHVATFKHSFNLLFDFKHEKIDTTNPIFYITLEGYFTPEEDADYIFGLQVFGTGVLYLDDELLIDQRKGQVSGDFCFGAGTIEKTKTVTLQKGKAYKVRIEYGSGPTSELVSEFGSGALQVGVTKAIDADEEIKKAAKLAAAHDKAILCIGLNAEWESEGHDREDMTLPARTNDLVRAVLEANPNTVIVNQSGTPVEFPWLQKANALVQAWYGGNELGNAIADVLYGDVVPNGKLSLSWPLKLEDNPAYLNFKTEFGRVVYGEDIFIGYRFYEKLQKRVAFPFGYGLSYTEFALSNLQVQINDEVISVSVDVKNTGEKYAGSEVVQVYIAATESSVSRAVKELKGFKKVLLQPGQTETAKIDLVLKDSVSFFDEEVGKWCSEAGQYKVLVGTSSDDIVLSESFDVEKTSYWSGL

SEQ ID NO: 4 - protein, 847 aa, Lachancea fermentati
MSKFDIEELIGELTLQEKIALIAAKDFWHTSPVERLGIPSVRVSDGPNGVRGTKFFNSVPSAAFPNGTGLASTFDTELLEEAGQLMAVEAAHKNASVILGPTTNIARGPLGGRGFESFSEDPYLSGMCTAALVNGMQSRGIAATVKHYVCNDLEDQRFSSNSIVTERALREIYLEPFRLAVKYANPVCIMSSYNKVNGTHCSQSKKLLDDILREEWGWDGMITSDWFGTYSSADAIKNGLDIEFPGPTKWRKSELISHLISSKEGISEEDVNTRVRNVLKMIKFAVDNKDKTGIIENGPESDANNTPETAAKLRKIAADSIVLLKNENNVLPLSKDESIVVIGPNAKTKFMSGGGSASLNPYYVVPIYDGIKSKLGKDPEYSIGCTSNKTLNGLWEACVIDPSKGNQADNIGAQAIFYTKPVEDRSPDEKPIDSTTIKQSYLTLFDYKNPAVEESNPLFYVDFEGYYTPEEDGDYEIGLQVYGTALLFIDGELVVDNKTKQTKGTFCFSAGTIEEKAIVSMKAGKSYKFRVEYGSGPTSQTASDFGAGGMQVGIAKKIDENEEIAHAAQLAKEHDKVVLCIGLNGEWESEGYDRENMTLPKKTNDLVRAVLRANPNTVVVNQSGTPVEMPWISECNALLQCWYGGNELGNAVADIAFADVVPSAKLSLSWPFKNEDNPAYLNFATESGRVLYGEDVFVGYRFYEKLQRQVAFPFGYGLSYTTFTFENLEVTADESKEILSVQLDVTNSGSKYAGAEVVQVYVAPTKSGITRPVKELKGFKKVYLEPNETKKVSLELPLKDSISYFEEYHNKWCAEAGEYQLLAGSSSDDTQLISSFELSKTFYWKGL

SEQ ID NO: 5 - protein, 845 aa, Saccharomyces mikatae
  source 1..845: note = strain: IFO 1815
MTFDIEKVLSELTTNEKISLIAAEDFWHTTPIKRLDIPSVRVSDGPNGIRGTKFFNSVPSAAFPNGTALASTFDKELLKEVGARMADEAIQKNAGVILGPTINIQRGPLGGRGFESFSEVPYLSGIAASCIVNGIQSRGVAATLKHFVCNDLEDQRMSSNSIVTCRALREIYLEPFKLAVKYSDPQCIMTSYNKVNGVHCSNSKNLLIDILRDEWKWGGMVMSDWFGTYSVDSIKNGLDIEFPGPSKWRSLDLLKSNLDSKAGITISNIDDCVRHVLKLVHYVSENSKKTQIKDHGPETTLNNTEHMSKHLRKVASESIVLLKNVDDILPLKKESSVVVIGPNAKAKSYSGGGSASLQPYYVITPYEGICEKIGRNVEYTAGCDSRKTLSGLIEAMVVDPCQPAEGDNIGIIAQYFMDPANQRSADVEPFDTHRVTQSYVTLFDYTHPNIDPIMPFFYIHFEGFFTPEEDGEYIFGVQVFGTALFYIDDKLEIDNKTHQTKGSFCFGAGTREETCEKYLVKGHQYRIRIEYGSGPTSSVAADFGYGGIQVGFAKKLNADEEIARAVQLAKTNDNVILCIGLNGEWESEGYDRENMSLPKNNDRLISAVLTANPNTIVVNQSGMPVELPWVDQCKALLQCWYGGNELGNAIADVLYGDVVPSGKLSISWPYMCHDNPAFLNFKTESGRVLYGEDIYVGYRFYEKVRRQVAFPFGHGLSYTTFRFDDLIVSIDETLDLLETSLNVTNTGDTIAGKEVIQVYVSHNESTIGRPIKELKGFQKVFLKPNETKKVTMKLSLKDSISYFDEEKQLWCATEGIYQILVGSSSKDIKLTERFDVNKTSYWKGL

SEQ ID NO: 6 - protein, 849 aa, Hanseniaspora osmophila
MPIDFNVDRLLTELTLDEKLSLLAGQDWWHTAAIERLNIPSVRVSDGPNGIRGTRFFACVPSACFPNGTALASTFNEEILESAGELMALEAKHKGAKVILGPTANIQRGPLGGRGFESFSEDPYLSGVATAAVVNGIQKSNEIAATVKHFVCNDMEHERFSSNSIVSERALREIYLEPFRLAVKHAQPKLFMTSYNKLNGIHCSSSKKLLQDILRNDWNSGATVISDWFGITDIVDSIQNGLDIEFPGPTRYRKPEILKNLLMCKTETREGGQFSIEHIDARARKVLELVKYFVEAEQSTDFPTNEDDHNNTFETAQFLRRLGNETIVLLKNETKLLPLDKKDDIVVIGPNAKAKNSSGGGCAALNGYYTISPLEGIANVTQRKTGDIPYTKGCDNHKNLSNLIEQCTNDADPEKKGAEMNFYTQPREVRGKEKPFDSYIIDQSFITLFDYKHEKVDEKKRLFYCTIEGYFIPKEDGDFEFQCQVLGTALFYIDDKLVINNKDDQTAGNFGFGSGTAPKNNIVTLEKGRKYKIFVDYGSGVTSKLSQSIAAGALQIGVNKVIDAEAEIKKAAELASKHDKVILVIGLNGEIESEGYDRDNMQLPRRTNDLVTAVLKANPNTVIVNQSGTPVEFPWLQQATTLLQAYYGGNELGNSIADVVFGDANPSGKLSLSWPLKNEDNPAYLNFKTVMGRVLYGEDIYVGYRFYEKLQRQVAYPFGHGLSYTTFKFNELDVSGDDESLKVELSVANTGKVDGKEVVQVYVARTSPSAVPRPVKELKKFKKVALKAGESAKVELTLSVKDSCSYFDEFHNQWHLEAGKYQVLVGSSSDDIHLIGDFEVKESEFWLGL

SEQ ID NO: 7 - protein, 837 aa, Clavispora sp.
  source 1..837: note = strain: NRRL Y-50464
MADIDVEKVLSELTLAEKIGLTAGVDFWHTYKVERLGVPTLRLSDGPNGVRGTKFVNGAPSACFPCGTGLASTFNKDLLYKAGRLMADEAKHKSAHVILGPTTNMQRGPLGGRGFESFSEDPHLAGMASASIVKGMQDNDIAATIKHFVCNDLEHERNSSDAIVTERALREIYLEPFRLAVKYADPKSFMTAYNKVNGEHVSQSHRILEQILREEWNWDGLVMSDWYGAYTAKESLTNGLDLEMPGPSGMRTVQNISHMVNSRELNIKYLDERVRNVLKLVKWCARSKLEERGPETTENNTPETRALLNKIASELVVLLKNNDSVLPLKKEESIAVIGPNAKFAAYCGGGSASLLSYYTTTPYDAIKEKLGHEPKYAVGCYAHQMLPGFSLSPYTKNPVTGKSGVNCKLYNDAPGTKNRRQFDEFDITMSPIILFDYRHPAIVDELFYMDITGDLTPEESGEYEFSLTVSGTAQLFIDDKLVIDNKNSQTLGTAFFGTGTIEMKQKVPLDAGKTYNVRVEFGSSKTSKLRPLSVISFGGCVSIGMCKVIDPKEEIAKAVELAKSVDKVVLLIGLNAEWESEGFDRPDMELPLLTNDLVEAVLAANPNTVVVNQSGTPVEMPWLSKANALVHAWYGGSEAGNAIANVLFGDVNPSGKLSLSWPFKNSDNPAYLNFHTERGRVLYGEDIYIGYRFYDKLQRRVAFPFGYGLSYTTYKYSDLNVTVNEEDDSLTASVTVENTGSKDGAETVQFYVAPKTSEVARPVKELKGFDKVFVKAGEKATAKVQLSLKDSASFFDEYHDKWSLEKGTYEVQVGKSSDDVELIQEFKVKESKLWSGL